(19) United States
(12) Patent Application Publication
(10) Pub. No.: US 2008/0181859 A1
(43) Pub. Date: Jul. 31, 2008

(54) Novel block (Co)polymers, compositions containing them, method of treatment and method of preparation

(76) Inventor: Celine Farcet, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/984,350

(22) Filed: Nov. 16, 2007

Related U.S. Application Data
(60) Provisional application No. 60/861,426, filed on Nov. 29, 2006.

(30) Foreign Application Priority Data
Nov. 16, 2006 (FR) 0654942

(51) Int. Cl. A61K 8/90; A61K 31/80; A61Q 5/00; A61Q 3/00; A61Q 19/00; C08F 275/00

(52) U.S. Cl. 424/59; 424/78.17; 424/70.12; 424/61; 424/78.03; 525/209

(57) ABSTRACT

Disclosed herein are novel block (co)polymers comprising from 35 to 100 wt. %, based on the weight of the block containing them, of silicone monomers chosen from a given list.

Also disclosed herein are cosmetic and pharmaceutical compositions containing said polymers, as well as a method of cosmetic treatment employing them, and a method of preparation of said polymers.

This application claims benefit of U.S. Provisional Application No. 60/861,426, filed Nov. 29, 2006, the contents of which are incorporated herein by reference. This application also claims benefit of priority under 35 U.S.C. § 119 to French Patent Application No. 0654942, filed Nov. 16, 2006, the contents of which are also incorporated herein by reference.

Disclosed herein are novel polymers of specific structure as well as cosmetic compositions containing said polymers. Also disclosed herein is a method of cosmetic treatment employing said polymers.

Various types of polymers are used conventionally in cosmetic compositions for the various properties that they can impart to them. They are used, for example, in compositions for make-up or care of the skin, of the lips or of the integumentary appendages, such as nail varnishes or compositions for the hair. However, if using, within a single composition, two polymers that are incompatible, i.e., immiscible in a single solvent, the incompatibility of the polymers may cause the formulator to face problems of separation of phases, or sedimentation, or the production of a composition that is not homogeneous. To date, these problems could most often only be solved if the composition contained a compound that was able to make the polymers compatible with one another.

To overcome this problem, European Patent Application No. 1 411 069 proposed polymers of a particular structure, comprising a first and at least one second block, incompatible with one another, and linked by an intermediate block which comprises at least one monomer that is a constituent of each of said two blocks.

EP 1 411 069 mainly describes block (co)polymers prepared from monomers such as acrylates and methacrylates of alkyl, including, for example, methyl, isobutyl, isobornyl, trifluoroethyl, and (meth)acrylic acid. These polymers may be carried in, and may be soluble in, organic solvents, including, for example, short esters, such as butyl and ethyl acetate, short alcohols, such as ethanol, and aliphatic alkanes, such as isododecane. They may be, however, quite difficult to carry in the silicone solvents usually employed in cosmetics.

Moreover, the polymers according to EP 1 411 069 give shiny films, which may not be suitable in certain cosmetic applications, for instance, in the area of foundations and certain compositions for skin care.

The present disclosure provides novel polymers, with which films may be produced that are more matt (less shiny), thus avoiding the usual addition of fillers to the composition to make it matt, and said polymers may moreover be carried both in carbon-. containing solvents and in silicone solvents, which was not previously possible.

Thus, disclosed herein is a block (co)polymer comprising at least one first block and at least one second block, wherein at least one of the blocks comprises, in an amount ranging from 35 to 100 wt. %, relative to the weight of said block, at least one silicone monomer chosen from the monomers described herein and mixtures thereof.

Also disclosed herein is a composition, which may be, for example, cosmetic and/or pharmaceutical, comprising said polymer in at least one physiologically acceptable medium.

Although EP 1 411 069 describes, in a general way, the possibility of incorporating additional silicone monomers in the block (co)polymer, nowhere does it describe, or even suggest, the presence of a large amount of silicone monomers, and even less the possibility of obtaining the remarkable properties found with the polymers according to the present disclosure.

The polymers disclosed herein therefore differ, in at least one embodiment, from those of the prior art by the presence of at least one silicone monomer, in a large amount, in at least one of the blocks.

The present inventors have found that the polymers disclosed herein may have good solubility in fats, whether, for example, carbon-containing or of the silicone type, and whether, for example, in cosmetic oils and solvents of the short ester type, and said solubility may vary and may be adjusted according to the nature and/or the quantity of monomers used. This good fat-solubility may facilitate their subsequent application, for instance in cosmetic compositions, which generally include a fatty phase.

Moreover, the polymers disclosed herein may make it possible to obtain matt films, without having to add fillers. These films may be less brittle than those of the prior art. They may also be less sticky when deposited and/or may be less sensitive to oils than those not comprising silicone monomers. They therefore may display better resistance to the ‘aggressive action’ of fats, such as oils in foods and/or sebum.

Moreover, the present inventors have found that the polymers disclosed herein may be more comfortable than those of the prior art containing little or no silicone monomers. The film may deform little or not at all during drying, thus avoiding problems of tightness.

Finally, the presence of silicone monomers may make it possible to obtain polymers with lower viscosity than the polymers of the prior art, which may make them easier to use in cosmetic compositions.

The polymers disclosed herein are block (co)polymers, comprising at least one first block and at least one second block, having different glass transition temperatures (Tg), said at least one first and at least one second blocks being advantageously linked together by an intermediate segment comprising at least one monomer that is a constituent of the first block and at least one monomer that is a constituent of the second block.

As used herein, the terms “first” and “second” blocks do not in any way define the order of said blocks (or sequences) in the structure of the polymer.

The block (co)polymer disclosed herein therefore comprises at least one first block and at least one second block, and in at least one embodiment, said blocks are incompatible with one another.

As used herein, the phrase “blocks that are incompatible with one another” is intended to mean that the mixture formed from the polymer corresponding to the at least one first block and from the polymer corresponding to the at least one second block is not miscible in the principal (by weight) polymerization solvent of the block (co)polymer, at room temperature (25° C.) and atmospheric pressure (10⁵ Pa), for a content of the mixture of polymers greater than or equal to 5 wt. %, relative to the total weight of the mixture (polymers and solvent), it being understood that:

i) said polymers are present in the mixture in an amount such that the respective weight ratio ranges from 10/90 to 90/10; and

ii) each of the polymers corresponding to the at least one first and at least one second blocks has an average molecular weight (weight-average or number-average molecular weight) equal to that of the block (co)polymer ±15%.

In the case of a mixture of polymerization solvents, assuming that at least two solvents are present, said mixture of polymers is immiscible in at least one of them. Of course, in the case of a polymerization carried out in a single solvent, the latter is the principal solvent.

In at least one embodiment, said first and second blocks may be linked together by an intermediate segment comprising at least one monomer m1 that is a constituent of the at least one first block and at least one monomer m2 that is a constituent of the at least one second block.

In one embodiment the intermediate segment forms a block (or sequence). In another embodiment, m2 is different from m1. In a further embodiment, the intermediate segment or block may enable said first and second blocks to be “compatibilized”.

In one embodiment, the block (co)polymer of the composition disclosed herein is an ethylenic block polymer, linear, branched or grafted. In a further embodiment, the block (co)polymer may form a deposit. In another embodiment it may be film-forming.

As disclosed herein “ethylenic” polymer is intended to mean a polymer obtained by polymerization of monomers comprising at least one ethylenic unsaturation.

As disclosed herein “block” polymer is intended to mean a polymer comprising at least 2 different blocks. In one embodiment the polymer comprises at least 3 different blocks.

As disclosed herein, the term polymer “forming a deposit” is intended to mean a polymer that is able to form on its own or in the presence of at least one additive, a deposit that adheres to a substrate, such as to keratinous substances.

As used herein, the term “film-forming” polymer is intended to mean a polymer that is able to form on its own or in the presence of at least one film-forming additive, a film that is continuous and adheres to a substrate, for example to keratinous substances.

Each block, or sequence, of the polymer according to the present disclosure is derived from one type of monomer or from several different types of monomers. This means that each block can be constituted of a homopolymer or of a copolymer, which may be random, alternating or of some other type.

In one embodiment, when it is present, the intermediate segment comprising at least one monomer that is a constituent of the first block and at least one monomer that is a constituent of the second block of the polymer is a random polymer. In a further embodiment, the intermediate block is derived essentially from monomers that are constituents of the first block and of the second block. As used herein, the term “essentially” is understood to mean at least 85%, for example at least 90%, at least 95% or at least 100%.

In at least one embodiment, the at least one first and at least one second blocks have different glass transition temperatures, with the difference between the glass transition temperatures of the first and second blocks generally being greater than 5° C., such as greater than 10° C., and, for example, greater than 20° C.

In one embodiment, the intermediate block has a glass transition temperature Tg between the glass transition temperatures of the at least one first and at least one second blocks.

The glass transition temperatures stated herein are, unless stated otherwise, theoretical Tg values determined from the theoretical Tg's of the monomers constituting each of the blocks, which can be found in a reference manual such as the Polymer Handbook, 4^{th} ed. (Brandrup, Immergut, Grulke), 1999, John Wiley, according to the following relation, called Fox's law:

$$\frac{1}{Tg} = {\sum }_{i} ( \frac{ϖ i}{Tgi} )$$

wherein ωi is the mass fraction of monomer i in the block under consideration and Tgi is the glass transition temperature of the homopolymer of monomer i (expressed in Kelvin).

The polymer disclosed herein is characterized in that at least one of its blocks comprises from 35 to 100 wt. %, relative to the total weight of said block, at least one silicone monomer chosen from, alone or mixed, the following monomers:

(i) ethylenic monomers wherein at least one ester group which comprises silanes and/or siloxanes, of formula:

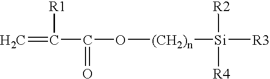

wherein:

R1 is chosen from H and methyl,

R2, R3, and R4, which may be identical or different, are chosen from C₁ to C₆ alkyl groups and —OSi(R5)₃ groups wherein R5 is chosen from methyl and ethyl; and in one embodiment R2, R3 and/or R4 are chosen, independently of one another, from —OSi(Me)₃ and/or methyl groups;

n is an integer ranging from 1 to 10, for example, equal to 1 or 3.

(ii) PDMS macromonomers and polydimethylsiloxanes comprising monoacryloyloxy and/or monomethacryloyloxy end groups, with the following formula

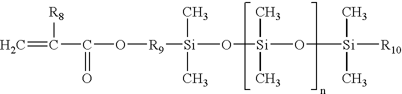

wherein:

R8 is chosen from hydrogen atoms and methyl groups;

R9 is chosen from linear or branched divalent hydrocarbon groups comprising from 1 to 10 carbon atoms; and in one embodiment, R9 contains one or two ether bonds —O—; and in another embodiment, R9 contains a group chosen from ethylene, propylene and butylene;

R10 is chosen from linear or branched alkyl groups, comprising from 1 to 10 carbon atoms, for example from 2 to 8 carbon atoms; and in one embodiment, R10 is chosen from methyl, ethyl, propyl, butyl and pentyl groups;

n is an integer ranging from 1 to 300, and in one embodiment, ranging from 3 to 200, and in another embodiment, ranging from 5 to 100.

(iii) ethylenic monomers wherein the ester group comprises at least one dendrimeric carboxysilane of formula:

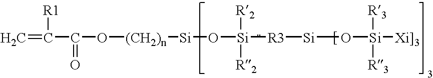

wherein:

R1 is chosen from H and methyl;

n is an integer ranging from 1 to 10, for example equal to 1, 2, 3 or 4;

R′2, R″2, R′3 and R″3, which may be identical or different, are chosen from C₁ to C₁₀ alkyl groups; and in one embodiment, are chosen from methyl and ethyl;

R3 is chosen from C₂ to C₁₀ divalent alkylene groups, and in one embodiment is chosen from C₂ and C₃;

i is an integer ranging from 1 to 10, for example equal to 1, 2 or 3; and

when i is an integer ranging from 2 to 10, X(i), which may be identical or different, is —R4-Si—[O—(R′3)(R″3)-X(i−1)]₃, wherein R4, which may be identical or different, is chosen from C₂ to C₁₀ divalent alkylene groups, for example C₂ or C₃;

when i is 1, X(1) is chosen from H and C₁ to C₁₀ alkyl groups, for example methyl and ethyl;

and in one embodiment, is chosen from the following monomers, wherein R1 is chosen from H and methyl:

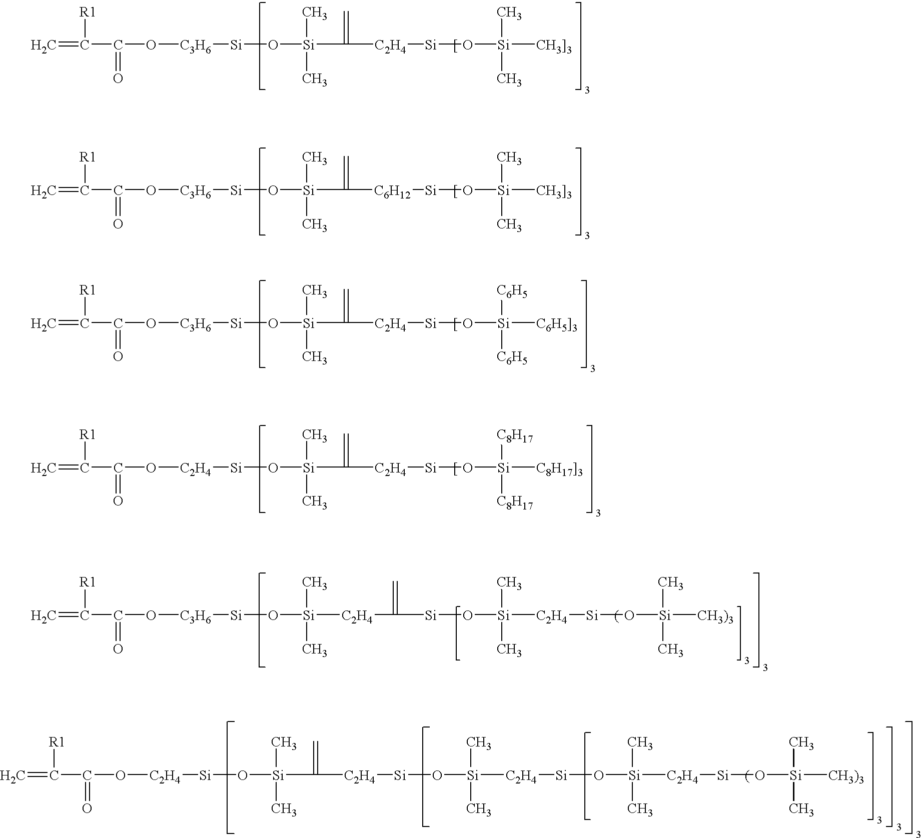

(iv) the ethylenic monomers of type POSS (polyhedral oligomeric silsesquioxanes) and POS (polyhedral polymeric silicates) of structure:

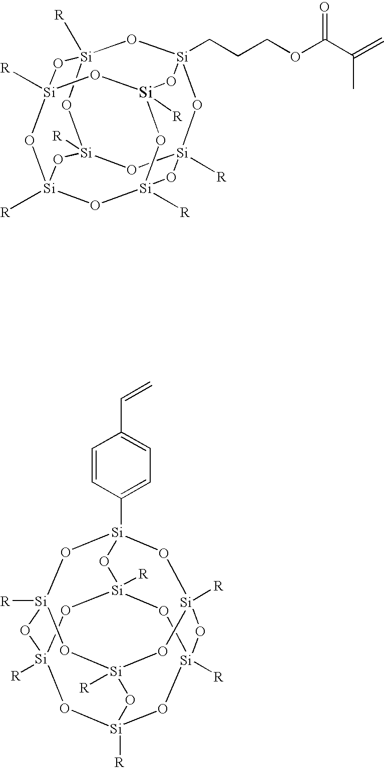

wherein R, which may be identical or different, is chosen from C₁ to C₁₀ linear alkyl groups, for example methyl, and C₃ to C₁₂ cyclic groups, for example C₅ cyclic groups; and

(v) mixtures thereof.

In one embodiment, the monomers of the type POSS and POS are chosen from: (meth)acryloxypropyltris(trimethylsiloxy)silane; (meth)acryloxypropyl-bis(trimethylsiloxy)methylsilane; (meth)acryloxymethyltris(trimethylsiloxy)-silane; (meth)acryloxymethylbis(trimethylsiloxy)methylsilane; (meth)acryl-oxypropyltrimethoxysilane; and, in at least one embodiment, the following monomers:

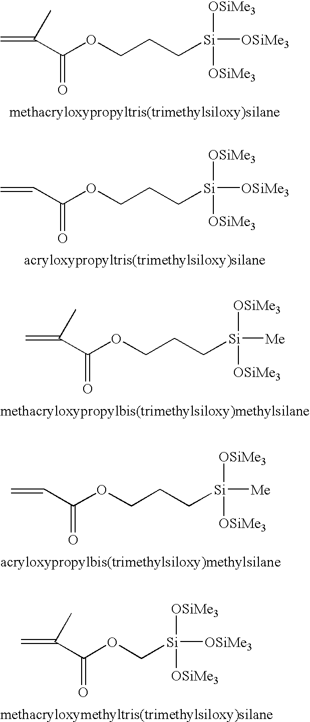

In another embodiment, (meth)acrylic monomers of type POSS (polyhedral oligomeric silsesquioxanes) and POS (polyhedral polymeric silicates) are chosen from Hybrid Plastics and the monomethacryloyloxypropyl polydimethylsiloxanes, such as those sold under the name PS560-K6 by UCT (United Chemical Technologies Inc.) and those sold under the name MCR-M17 by Gelest Inc.

In at least one embodiment, the aforesaid at least one silicone monomer may be present in an amount ranging from 35 to 100 wt. %, for instance ranging from 50 to 99 wt. %, or 60 to 95 wt. %, or 70 to 90 wt. %, relative to the total weight of the block containing the at least one silicone monomer.

In another embodiment, the at least one silicone monomer is present in an amount ranging from 20 to 90 wt. % relative to the total weight of the polymer, such as from 25 to 80 wt. % or, for example, 30 to 75 wt. %, such as from 35 to 70 wt. %, relative to the total weight of the polymer.

In one embodiment, it may be possible to obtain at least one polymer that is soluble in silicone fatty phases, for example in D5 and/or phenyltrimethicone, at 25° C., 1 atm., and at a content of 1 wt. % or 10 wt. % relative to the total weight.

As used herein, “soluble” is intended to mean that the polymer forms a clear solution, without aggregates or sediments.

In at least one embodiment, the at least one silicone monomer is present, alone or as a mixture, in the at least one second block (formed secondly). In another embodiment the at least one silicone monomer may be present in the at least one first block. In yet another embodiment, the at least one silicone monomer may be present in at least one first and at least one second blocks. In yet another embodiment, the at least one silicone monomer may be present in all of the blocks of the polymer. In one embodiment, the at least one silicone monomer is present in the intermediate block. In another embodiment, the at least one silicone. monomer is not present in the intermediate block.

In at least one embodiment, the at least one silicone monomer is present in at least one block with low Tg, for example a block wherein Tg is less than or equal to 20° C. In another embodiment Tg is less than or equal to 0° C., such as less than or equal to −10° C.

In at least one embodiment, the polymer disclosed herein is soluble in the carbon-containing fatty phases, for example in oils such as isododecane and/or Parleam, at 25° C., 1 atm., at a content of 1 wt. % or 10 wt. % relative to the total weight of the composition.

In at least one embodiment the polymer may comprise at least one carbon-containing monomer present in an amount ranging from 10 to 80 wt. % of the total weight, such as from 20 to 75 wt. %, or, for example, from 25 to 70 wt. %, such as from 30 to 65 wt. %, relative to the total weight of the polymer.

In one embodiment, the at least one carbon-containing monomer may be present in the same block as the silicone monomers. In another embodiment, the at least one carbon-containing monomer is present in a separate block.

In at least one embodiment, the at least one carbon-containing monomer is chosen from:

methacrylates of formula CH₂═C(CH₃)—COOR1

wherein R1 is chosen from: linear and branched C₈ to C₂₂ alkyl groups, such as lauryl, behenyl or stearyl groups; and cyclic alkyl groups comprising from 8 to 30 carbon atoms, such as isobornyl; and tert-butyl groups;

acrylates of formula CH₂═CH—COOR2

wherein R2 is chosen from: linear or branched C₈ to C₂₂ alkyl groups, such as lauryl, behenyl, stearyl or 2-ethylhexyl; and cyclic alkyl groups comprising 8 to 30 carbon atoms, such as isobornyl; and isobutyl groups;

(meth)acrylamides of formula CH₂═C(CH₃)—CONR3R4 or CH₂═CH—CONR3R4,

wherein R3 is chosen from hydrogen and linear and branched C₁ to C₁₂ alkyl groups, and R4 is chosen from linear and branched C₈ to C₁₂ alkyl groups, such as isooctyl, isononyl, and undecyl groups;

di-n-alkyl itaconates of formula CH₂═C(CH₂—COO(CH₂)ₙ—CH₃)COO(CH₂)ₙ—CH₃, wherein n is an integer greater than or equal to 5, for example ranging from 6 to 13;

vinyl esters of formula CH₂═CHOCOR5 wherein R5 is chosen from linear or branched C₈ to C₂₂ alkyl groups;

vinyl ethers of formula CH₂═CH—OR6 wherein R6 is chosen from linear and branched alkyl groups, comprising from 8 to 22 carbon atoms;

carbon-containing macromonomers comprising a polymerizable end group;

and mixtures thereof.

As used herein, “macromonomer comprising a polymerizable end group” is intended to mean any oligomer comprising, at just one of its ends, at least one polymerizable end group that is able to react during the reaction of polymerization with ethylenic monomers. In one embodiment, the at least one polymerizable group of the macromonomer may be a group with an ethylenic unsaturation that can undergo radical polymerization. In one embodiment, the at least one polymerizable end group is chosen from vinyl, (meth)acrylate and (meth)acryloxy groups. In a further embodiment the at least one polymerizable end group is a (meth)acrylate group.

As used herein, “carbon-containing macromonomer” is intended to mean a non-silicone macromonomer, such as, for example an oligomeric macromonomer obtained by polymerization of at least one non-silicone monomer with at least one ethylenic unsaturation. In a further embodiment, the at least one carbon-containing macromonomer may be obtained by polymerization of acrylic and/or vinylic non-acrylic monomers.

Examples of carbon-containing macromonomers comprising at least one polymerizable end group include, but are not limited to:

(i) linear or branched C₆ to C₂₂ groups, for example, C₈ to C₁₁ alkyl (meth)acrylate homopolymers and copolymers, comprising at least one polymerizable end group chosen from vinyl and (meth)acrylate groups, including, for example, poly(ethyl-2 hexyl acrylate) macromonomers with at least one mono(meth)acrylate end group; poly(dodecyl acrylate) or poly(dodecyl methacrylate) macromonomers with at least one mono(meth)acrylate end group; and poly(stearyl acrylate) or poly(stearyl methacrylate) macromonomers with at least one mono(meth)acrylate end group. Such macromonomers are described, for example, in European Patent Nos. 895 467 and 096 459 and in the article Gillman, Polymer Letters, Vol 5, page 477-481 (1967).

In one embodiment, the macromonomers may be chosen from those based on poly(ethyl-2-hexyl acrylate) or on poly(dodecyl acrylate) with a mono(meth)acrylate end group.

(ii) polyolefins comprising at least one end group with an ethylenic unsaturation, for example those comprising at least one (meth)acrylate end group. Examples of polyolefins comprising at least one (meth)acrylate end group include, but are not limited to: macromonomers of polyethylene; macromonomers of polypropylene; macromonomers of polyethylene/polypropylene copolymer; macromonomers of polyethylene/polybutylene copolymer;. macromonomers of polyisobutylene; macromonomers of polybutadiene; macromonomers of polyisoprene; macromonomers of polybutadiene; and macromonomers of poly(ethylene/butylene)-polyisoprene. Such macromonomers are described, for example, in European Patent No. 1 347 013 and in U.S. Pat. No. 5,625,005 which mention ethylene/butylene and ethylene/propylene macromonomers with a (meth)acrylate reactive end group. A non-limiting example of such macromers is poly(ethylene/butylene) methacrylate, such as the product sold under the name Kraton Liquid L-1253 by Kraton Polymers.

In one embodiment, the at least one carbon-containing monomer is chosen from: lauryl, behenyl, stearyl, isobornyl, and tert-butyl methacrylates; lauryl, behenyl, 2-ethylhexyl, stearyl, isobornyl, and isobutyl acrylates; macromonomers based on poly(ethyl-2-hexyl acrylate) and/or poly(dodecyl acrylate) comprising a mono(meth)acrylate end group; ethylene/butylene and ethylene/propylene macromonomers comprising a (meth)acrylate reactive end group; and mixtures thereof.

When the at least one silicone monomer does not represent 100 wt. % relative to the weight of its block, the block may include at least one additional monomer, which may be chosen from at least one carbon-containing monomer and/or at least one additional monomer, and which may be present in an amount ranging from 0.1 to 65 wt. % relative to the total weight of the block, for example from 1 to 50 wt. %, or from 5 to 40 wt. %, or from 10 to 30 wt. %.

In another embodiment, the block comprising said at least one silicone monomer may be a homopolymer comprising a single silicone monomer; a copolymer comprising several silicone monomers described above, or a copolymer comprising at least one silicone monomer described above and at least one other monomer (chosen, in one embodiment, from additional carbon-containing monomers). When at least one block is a copolymer, the at least one block may comprise monomers that can be, as already mentioned, positioned randomly, alternating or of some other type relative to the other copolymer components.

Similarly, when the at least one carbon-containing monomer chosen does not represent 100 wt. % of their block, the block may include at least one silicone monomer and/or at least one additional monomer that may be identical to or different from the additional monomers of the silicone block, and which may be present in an amount ranging from 0.1 to 65 wt. % relative to the total weight of the block, for example from 1 to 50 wt. %, or 5 to 40 wt. %, or 10 to 30 wt. %; and wherein the at least one carbon-containing monomer chosen may be present in an amount ranging from 35 to 99.9 wt. % relative to the total weight of the block, for example from 50 to 99 wt. %, or from 60 to 95 wt. %, or from 70 to 90 wt. %.

Examples of the at least one additional monomer include, but are not limited to the following monomers:

(i) ethylenic hydrocarbons having 2 to 10 carbon atoms, such as ethylene, isoprene, or butadiene;

(ii) (meth)acrylates chosen from compounds of formulas CH₂═CHCOOR′₃ and CH₂═C(CH₃)COOR′₃ wherein R′₃ is chosen from:

linear or branched alkyl groups, comprising from 1 to 7 carbon atoms, for example from 4 to 7 carbon atoms, said alkyl groups being optionally interrupted by at least one heteroatom chosen from O, N, S and P and optionally substituted with at least one substituent chosen from hydroxyl groups and halogen atoms, for example Cl, Br, I and F;

and in one embodiment R′3 may be chosen from: methyl, ethyl, propyl, n-butyl, isobutyl, tert-butyl, and hexyl groups; ethyl-2-perfluorohexyl groups; C₁₋₄ hydroxyalkyl groups, such as 2-hydroxyethyl, 2-hydroxybutyl and 2-hydroxypropyl groups; and alkoxy (C₁₋₄) alkyl (C₁₋₄) groups, such as methoxyethyl, ethoxyethyl and methoxypropyl groups;

C₃ to C₇ cycloalkyl groups, such as the cyclohexyl groups;

C₃ to C₂₀ aryl groups, such as the phenyl groups;

C₄ to C₃₀ aralkyl groups (e.g., C₁ to C₈ alkyl groups), such as 2-phenylethyl, t-butylbenzyl or benzyl groups;

heterocyclic groups comprising from 4 to 12 ring members, comprising at least one heteroatom chosen from O, N, and S, wherein the ring may optionally be aromatic;

heterocycloalkyl (C₁ to C₄ alkyl) groups, such as furfurylmethyl or tetrahydrofurfurylmethyl groups,

where said cycloalkyl, aryl, aralkyl, heterocyclic or heterocycloalkyl groups may optionally be substituted with at least one substituent chosen from hydroxyl groups, halogen atoms, and linear or branched C₁₋₄ alkyl groups, said alkyl groups being optionally interrupted by at least one heteroatom chosen from O, N, S and P, and optionally substituted with at least one substituent chosen from hydroxyl groups and halogen atoms, for example Cl, Br, I and F;

—(C₂H₄O)ₘ—R″ groups, wherein m is an integer ranging from 5 to 150 and R″ is chosen from H and alkyl groups comprising from C₁ to C₃₀, for example -POE-methyl or -POE-behenyl groups; and

isobutyl methacrylate and tert-butyl acrylate;

(iii) (meth)acrylamides of formula:

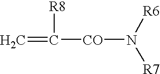

wherein R₈ is chosen from H and methyl groups; and R₇ and R₆, which may be identical or different, are chosen from:

hydrogen atoms;

linear or branched alkyl groups comprising, from 1 to 22 carbon atoms, for example, from 13 to 20, or from 14 to 18 carbon atoms, said alkyl groups being optionally interrupted by at least one heteroatom chosen from O, N, S and P; and optionally substituted with at least one substituent chosen from hydroxyl groups and halogen atoms. for example Cl, Br, I and F; and R6, and/or R7 may be, for example, a methyl, ethyl, propyl, n-butyl, isobutyl, tert-butyl, hexyl, ethylhexyl, octyl, lauryl, isooctyl, isodecyl, dodecyl, behenyl, and/or stearyl groups; ethyl-2-perfluorohexyl groups; C₁₋₄ hydroxyalkyl groups, such as 2-hydroxyethyl, 2-hydroxybutyl and/or 2-hydroxypropyl; and/or alkoxy (C₁₋₄) alkyl (C₁₋₄) groups such as methoxyethyl, ethoxyethyl and/or methoxypropyl,

C₃ to C₁₂ cycloalkyl groups, such as isobornyl, cyclohexyl, and t-butylcyclohexyl groups;

C₃ to C₂₀ aryl groups, such as phenyl,

C₄ to C₃₀ aralkyl groups (C₁ to C₈ alkyl groups), such as 2-phenyl-ethyl, t-butylbenzyl and benzyl,

heterocyclic groups comprising from 4 to 12 ring members comprising at least one heteroatom chosen from O, N, and S, and in one embodiment the ring may optionally be aromatic,

heterocycloalkyl groups (C₁ to C₄ alkyl), such as furfurylmethyl or tetrahydrofurfurylmethyl,

and in at least one embodiment said cycloalkyl, aryl, aralkyl, heterocyclic or heterocycloalkyl groups may optionally be substituted with at least one substituent chosen from: hydroxyl groups; halogen atoms; and linear or branched C₁ to C₄ alkyl groups optionally interrupted by at least one heteroatom chosen from O, N, S and P and optionally substituted with at least one substituent chosen from hydroxyl groups and halogen atoms, for example Cl, Br, I and/or F.

Examples of (meth)acrylamide monomers include but are not limited to (meth)acrylamide, N-ethyl(meth)acrylamide N-butylacrylamide, N-t-butylacrylamide N-isopropylacrylamide, N,N-dimethyl(meth)acrylamide, N,N-dibutylacrylamide, and N(2-hydroxypropylmethacrylamide).

(iv) vinylic compounds of formulae:

CH₂═CH—R₉, CH₂═CH—CH₂—R₉ or CH₂═C(CH₃)—CH₂—R₉

wherein R₉ is chosen from: hydroxyl groups; halogen groups, such as Cl or F; NH₂; OR₁₄ groups, wherein R₁₄ is chosen from phenyl groups and C₁ to C₇ alkyl groups; acetamide (NHCOCH₃); OCOR₁₅ groups, wherein R₁₅ is chosen from linear and branched alkyl groups comprising from 2 to 7 carbon atoms; and groups chosen from:

linear and branched alkyl groups, comprising from 1 to 22 carbon atoms, for example from 4 to 20, or from 6 to 18 carbon atoms, said alkyl groups being optionally interrupted by at least one heteroatom chosen from O, N, S and P; and optionally substituted with at least one substituent chosen from hydroxyl groups and halogen atoms, for example Cl, Br, I and/or F;

C₃ to C₁₂,cycloalkyl groups, such as isobornyl and cyclohexyl;

C₃ to C₂₀ aryl groups, such as phenyl;

C₄ to C₃₀ aralkyl groups (C₁-C₈ alkyl groups), such as 2-phenylethyl and benzyl;

heterocyclic groups comprising from 4 to 12 ring members and further comprising at least one heteroatom chosen from O, N, and S, wherein the ring may optionally be aromatic;

and heterocycloalkyl (C₁ to C₄ alkyl) groups, such as furfurylmethyl and tetrahydrofurfurylmethyl,

and said cycloalkyl, aryl, aralkyl, heterocyclic or heterocycloalkyl groups may optionally be substituted with at least one substituent chosen from: hydroxyl groups; halogen atoms; and linear and branched alkyl groups comprising from 1 to 4 carbon atoms, said alkyl groups being optionally interrupted by at least one heteroatom chosen from O, N, S and P, and optionally substituted with at least one substituent chosen from hydroxyl groups and halogen atoms, for example Cl, Br, I and/or F.

Examples of vinylic monomers include, but are not limited to, vinylcyclohexane, and styrene. Examples of vinyl esters include, but are not limited to, vinyl acetate, vinyl propionate, and vinyl butyrate. Examples of vinyl ethers include, but are not limited to, vinyl methyl ether, vinyl ethyl ether and vinyl isobutyl ether.

(v) monomers comprising at least one ethylenic unsaturation, and further comprising at least one carboxylic, phosphoric or sulphonic acid, or anhydride function, for example, acrylic acid, methacrylic acid, crotonic acid, maleic anhydride, itaconic acid, fumaric acid, maleic acid, acrylamidopropanesulphonic acid, vinylbenzoic acid, vinylphosphoric acid and salts thereof;

(vi) monomers comprising at least one ethylenic unsaturation, and further comprising at least one tertiary amine function, such as 2-vinylpyridine, 4-vinylpyridine, dimethylaminoethyl methacrylate, diethylaminoethyl methacrylate, dimethylaminopropyl methacrylamide and salts thereof;

(vii) and mixtures thereof.

In at least one embodiment, the aforementioned salts may be formed by neutralization of the anionic groups with at least one inorganic base, such as LiOH, NaOH, KOH, Ca(OH)₂, NH₄OH and/or Zn(OH)₂; and/or with at least one organic base such as a primary, secondary or tertiary alkyl amine, such as triethylamine and/or butylamine. In one embodiment, at least one primary, secondary or tertiary alkyl amine may contain at least one nitrogen atom and/or oxygen atom, and may therefore contain, for example, at least one alcohol function; In a further embodiment, the at least one organic base is chosen from amino-2-methyl-2-propanol, triethanolamine and dimethylamino-2-propanol. In yet another embodiment, the at least one organic base is chosen from lysine and 3-(dimethylamino)-propylamine.

Examples of salts of the at least one inorganic acid disclosed herein include, but are not limited to, the salts of sulphuric acid, hydrochloric acid, hydrobromic acid, hydriodic acid, phosphoric acid, and/or boric acid. Examples of salts of at the at least one organic acids disclosed herein include, but are not limited to salts comprising at least one carboxylic, sulphonic, and/or phosphonic acid group. In one embodiment, the acids disclosed herein may be linear, branched, and/or cyclic aliphatic acids. In a further embodiment, they may be aromatic acids. In yet another embodiment, these acids may contain at least one heteroatom chosen from O and N, for example in the form of hydroxyl groups. In a further embodiment, the acids are chosen from propionic acid, acetic acid, terephthalic acid, citric acid and tartaric acid.

In one embodiment, the at least one additional monomer is chosen from (meth)acrylic acid and from C₁ to C₄ alkyl and from C₃ to C₇ cycloalkyl (meth)acrylates. In a further embodiment, the at least one additional monomer is chosen from methyl methacrylate, isobutyl methacrylate, tert-butyl acrylates, acrylic acid and methacrylic acid, or a mixture thereof.

In one embodiment, the at least one additional monomer may be present in an amount ranging from 0 to 70 wt. % relative to the total weight of the polymer, such as from 0.1 to 30 wt. % or, for example, from 0.5 to 20 wt. %, such as from 1 to 10 wt. %.

In at least one embodiment, the silicone monomers are present in a different block from the one containing the chosen carbon-containing monomers.

In one embodiment, the polymer disclosed herein may, in addition, comprise other blocks, which may be of any chemical nature.

In at least one embodiment, the polymer disclosed herein includes a block with Tg greater than or equal to 20° C., for example between 20° C. and 160° C., or between 30° C. and 140° C., or between 40° C. and 120° C., or between 50° C. and 110° C., and may comprise at least one carbon-containing monomer.

In one embodiment, the polymer disclosed herein comprises at least one block wherein Tg is below 20° C., for example between −150° C. and 20° C. exclusively, or between −100° C. and 10° C., or between −85° C. and 0° C., or between −70° C. and −5° C. In a further embodiment, the polymer having Tg below 20° C. comprises at least one silicone monomer.

In at least one embodiment, at least one block with Tg greater than or equal to 20° C. is present in an amount ranging from 50 to 90 wt. % relative to the total weight of the polymer, for example from 60 to 80 wt. %, or 65 to 75 wt. %.

In one embodiment, the at least one block with Tg greater than or equal to 20° C. may comprise at least one monomer comprising at least one homopolymer having a Tg greater than or equal to 20° C. In a further embodiment, it may also comprise at least one monomer having a Tg outside of this range. These monomers and their concentrations may be chosen appropriately by a person skilled in the art, for example on the basis of Fox's law, to obtain a block with the desired Tg. Examples of monomers having a Tg greater than or equal to 20° C. include, but are not limited to:

methacrylates of formula: CH₂═C(CH₃)—COOR₁ wherein R₁ is chosen from linear and branched, unsubstituted alkyl groups, comprising from 1 to 4 carbon atoms, such as methyl, ethyl, propyl or isobutyl groups, or alternatively R₁ comprises a C₄ to C₁₂ cycloalkyl group, for example an isobornyl group;

acrylates of formula: CH₂═CH—COOR₂ wherein R₂ is chosen from tert-butyl groups and C₄ to C₁₂ cycloalkyl groups, such isobornyl groups;

(meth)acrylamides of formula: CH₂═CR′—CO—NR⁷R⁸ wherein R′ is chosen from H and CH₃, and R⁷ and R⁸, which may be identical or different, are chosen from hydrogen atoms and linear and branched C₁ to C₁₂ alkyl groups, such as an n-butyl, t-butyl, isopropyl, isohexyl, isooctyl, or isononyl groups; or in an alternate embodiment, R⁷ comprises H and R⁸ comprises a 1,1-dimethyl-3-oxobutyl group,

methacrylic acid and/or acrylic acid

and/or mixtures thereof.

Examples of monomers comprising homopolymers having a glass transition temperature Tg greater than or equal to 20° C. include, but are not limited to, methyl, methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide, and N,N-dibutylacrylamide and mixtures thereof.

In one embodiment, the at least one block having a Tg below 20° C. may comprise monomers whose homopolymers have a Tg below 20° C. In another embodiment, it may comprise monomers having a Tg outside of this range. These monomers and their concentration may be chosen appropriately by a person skilled in the art, for example on the basis of Fox's law, to obtain a block with the desired Tg. Examples of monomers having a Tg below 20° C. include, but are not limited to:

acrylates of formula CH₂═CHCOOR₃, wherein R₃ is chosen from linear and branched from C₁ to C₁₂ unsubstituted alkyl groups, with the exception of the tert-butyl group, optionally interrupted by at least one heteroatom chosen from O, N, and S,

methacrylates of formula CH₂═C(CH₃)—COOR₄, wherein R₄ is chosen from linear and branched from C₆ to C₁₂ unsubstituted alkyl groups, optionally interrupted by at least one heteroatom chosen from O, N and S;

vinyl esters of formula R₅—CO—O—CH═CH₂ wherein R₅ is chosen from linear and branched C₄-C₁₂ alkyl groups;

—(C₄-C₁₂ alkyl)-vinylethers, for example methylvinylether and ethylvinylether;

N—(C₄-C₁₂ alkyl)acrylamides, such as N-octylacrylamide,

and mixtures thereof.

Nonlimiting examples of monomers with Tg below 20° C. include methyl acrylate, ethyl acrylate, isobutyl acrylate, ethyl-2-hexyl (meth)acrylate and mixtures thereof.

In one embodiment, the polymer disclosed herein comprises, in at least one block, at least one monomer chosen from esters of (meth)acrylic acid. In a further embodiment optionally, it may additionally include at least one second monomer chosen from acrylic acid and methacrylic acid, and mixtures thereof.

In at least one embodiment, all the monomers other than the silicone monomers are chosen from (meth)acrylic acid and esters of (meth)acrylic acid.

In one embodiment, the weight-average molar mass (Mw) of the polymer disclosed herein ranges from 25,000 to 1,000,000, for example from 30,000 to 750,000, or from 40,000 to 500,000, or from 50,000 to 250,000.

The weight-average (Mw) and number-average (Mn) molar masses may be determined by gel-permeation liquid chromatography, for example using the solvent THF, and the calibration curve may be established with standards of linear polystyrene, and using refractometric and UV detectors.

In at least one embodiment, the polydispersity index of the polymer disclosed herein is greater than 2, for example in the range from 2 to 9, or greater than or equal to 2.5, for example in the range from 2.5 to 8, or in the range from 2.8 to 7. As used herein, the “polydispersity index” (Ip) of the polymer is equal to the ratio of the weight-average molar mass (Mw) to the number-average molar mass (Mn).

In one embodiment, the polymer disclosed herein may be obtained by radical solution polymerization according to the following method of preparation:

load a portion of the polymerization solvent in a suitable reactor, and heat until a suitable temperature for polymerization is reached (typically between 60 and 120° C.),

once this temperature has been reached, the monomers constituting the first block can be added, in the presence of a portion of the polymerization initiator,

after a time T corresponding to a maximum degree of conversion of, for example, 90%, the monomers constituting the second block and the other portion of the initiator can be introduced,

leave the mixture to react for a time T′ (for example, ranging from 3 to 6 hours) at the end of which the mixture is brought back to room temperature (25° C.),

so as to obtain the polymer in solution in the polymerization solvent.

As used herein, “polymerization solvent” is intended to mean a solvent, or a mixture of solvents, for example those chosen from ethyl acetate, butyl acetate, from C₁ to C₆ alcohols such as isopropanol, ethanol, aliphatic alkanes such as isododecane, and mixtures thereof. In at least one embodiment, the polymerization solvent is a mixture of butyl acetate and isopropanol or is isododecane, and in another embodiment it is D5 (cyclopentadimethylsiloxane).

In at least one embodiment, the polymer disclosed herein is not water-soluble, i.e., the polymer is not soluble in water or in a mixture of water and linear or branched monoalcohols comprising from 2 to 5 carbon atoms such as ethanol, isopropanol or n-propanol, without change of pH, and wherein the content of active substance is at least 1 wt. % relative to the total weight of the composition, at room temperature (25° C.).

Also disclosed herein are compositions, which may be cosmetic or pharmaceutical, comprising at least one polymer of specific structure as described above.

In at least one embodiment, said polymer is in solution in the physiologically, e.g., cosmetically or pharmaceutically, acceptable medium of said compositions. In one embodiment, said polymer is in solution in at least one fatty phase of said compositions, for example in an oil and/or a carbon-containing and/or silicone organic solvent.

In one embodiment, the polymers disclosed herein may be present, alone or as a mixture, in the compositions disclosed herein in an amount ranging from 0.01 to 50 wt. % relative to the total weight of the composition, for example from 0.05 to 40 wt. %, or from 0.1 to 30 wt. %, or from 0.5 to 20 wt. %, or from 1 to 15 wt. %, or from 1.5 to 12 wt. %.

In one embodiment, the cosmetic or pharmaceutical compositions disclosed herein comprise, in addition to said polymers, at least one physiologically acceptable medium, for example a medium that is cosmetically and/or pharmaceutically acceptable. As used herein, a medium that is cosmetically and/or pharmaceutically acceptable includes, for example, a medium that is compatible with keratinous substances such as the skin of the face or of the body, the hair, the eyelashes, the eyebrows and the nails.

In one embodiment, the composition may comprise at least one hydrophilic medium comprising water or a mixture of water and at least one hydrophilic organic solvent such as alcohols, for example: the linear and branched lower monoalcohols comprising from 2 to 5 carbon atoms, such as ethanol, isopropanol or n-propanol; polyols, such as glycerol, diglycerol, propylene glycol, sorbitol, pentylene glycol; polyethylene glycols; and hydrophilic C₂ ethers and from C₂ to C₄ aldehydes.

In one embodiment, the water or the mixture of water and hydrophilic organic solvents may be present in the composition disclosed herein at a content in the range from 0.1 to 99 wt. %, relative to the total weight of the composition, for example from 10 to 80 wt. %.

In one embodiment, the composition may also be anhydrous.

In another embodiment, the composition may also comprise at least one fatty phase which may comprise at least one fat that is liquid at room temperature (25° C.) and/or at least one fat that is solid at room temperature, such as waxes, pasty fats, gums and mixtures thereof. In yet another embodiment, the at least one fat may be of animal, vegetable, mineral or synthetic origin. In a further embodiment, said at least one fatty phase may comprise lipophilic organic solvents.

Examples of fats that are liquid at room temperature, often called oils, include but are not limited to: hydrocarbon oils of animal origin such as perhydrosqualene; vegetable hydrocarbon oils such as liquid triglycerides of fatty acids comprising from 4 to 10 carbon atoms, such as triglycerides of heptanoic or octanoic acids, or sunflower oil, maize oil, soya oil, grapeseed oil, sesame oil, apricot oil, macadamia oil, castor oil, avocado oil, triglycerides of caprylic/capric acids, jojoba oil, shea butter oil; linear or branched hydrocarbons of mineral or synthetic origin, such as paraffin oils and their derivatives, vaseline, polydecenes, hydrogenated polyisobutene such as Parleam; synthetic esters and ethers, such as those of fatty acids, for example purcelline oil, isopropyl myristate, ethyl-2-hexyl palmitate, octyl-2-dodecyl stearate, octyl-2-dodecyl erucate, and isostearyl isostearate; hydroxylated esters such as isostearyl lactate, octylhydroxystearate, octyldodecyl hydroxystearate, diisostearylmalate, triisocetyl citrate, heptanoates, octanoates, and decanoates of aliphatic alcohols; polyol esters such as propylene glycol dioctanoate, neopentylglycol diheptanoate, and diethyleneglycol diisononanoate; esters of pentaerythritol; aliphatic alcohols comprising from 12 to 26 carbon atoms, such as octyldodecanol, 2-butyloctanol, 2-hexyldecanol, 2-undecylpentadecanol, and oleic alcohol; partially fluorinated hydrocarbon and/or silicone oils; silicone oils such as polymethylsiloxanes (PDMS) that are volatile or non-volatile, linear or cyclic, and liquid or pasty at room temperature, such as cyclomethicones, dimethicones which may optionally comprise at least one phenyl group, such as phenyl trimethicones, phenyltrimethylsiloxydiphenyl siloxanes, diphenylmethyldimethyl-trisiloxanes, diphenyl dimethicones, phenyl dimethicones, and polymethylphenyl siloxanes; and mixtures thereof.

In at least one embodiment, the oils disclosed herein may be present in an amount ranging from 0.01 to 90%, for example from 0.1 to 85 wt. %, relative to the total weight of the composition.

In one embodiment, the composition disclosed herein may comprise at least one physiologically acceptable organic solvent.

In at least one embodiment, the solvents disclosed herein may be present in an amount ranging from 0.1 to 90% relative to the total weight of the composition, for example from 0.5 to 85%, or from 10 to 80 wt. %, or from 30 to 50%.

In addition to the hydrophilic organic solvents mentioned previously examples of hydrophilic organic solvents include, but are not limited to: the ketones that are liquid at room temperature, such as methyl ethyl ketone, methyl isobutyl ketone, diisobutyl ketone, isophorone, cyclohexanone, and acetone; ethers of propylene glycol that are liquid at room temperature, such as monomethylether of propylene glycol, acetate of monomethyl ether of propylene glycol, and mono n-butyl ether of dipropylene glycol; short-chain esters comprising from 3 to 8 carbon atoms, such as ethyl acetate, methyl acetate, propyl acetate, n-butyl acetate, and isopentyl acetate; ethers that are liquid at 25° C., such as diethylether, dimethylether and dichlorodiethylether; alkanes that are liquid at 25° C. such as decane, heptane, dodecane, isododecane, and cyclohexane; aromatic cyclic compounds that are liquid at 25° C., such as toluene and xylene; aldehydes that are liquid at 25° C., such as benzaldehyde, acetaldehyde; and mixtures thereof.

As used herein, “wax” is intended to mean a lipophilic compound, which is solid at room temperature (25° C.), and has a reversible solid/liquid change of state, and having a melting point above or equal to 25° C. and up to 120° C. In one embodiment, on bringing at least one wax to the liquid state (fusion), it may be possible to make it miscible with at least one oil optionally present, and to form a microscopically homogeneous mixture, but on lowering the temperature of said mixture to room temperature, the at least one wax undergoes recrystallization in the oils of the mixture. The melting point of the at least one wax may be measured using a differential scanning calorimeter (D.S.C.), for example the calorimeter sold under the name DSC 30 by the METLER company.

In at least one embodiment, at least one wax is chosen from hydrocarbon, fluorinated, and/or silicone waxes, and can be of vegetable, mineral, animal and/or synthetic. origin. In one embodiment, the at least one wax has a melting point above 30° C. In another embodiment, the melting point is above 45° C. Examples of wax for use in the composition disclosed herein include, but are not limited to: beeswax; carnauba wax or candelilla wax; paraffin wax; microcrystalline waxes; ceresine wax or ozokerite; synthetic waxes, such as polyethylene waxes or Fischer-Tropsch waxes; and silicone waxes such as alkyl or alkoxy dimethicone comprising from 16 to 45 carbon atoms.

Examples of gums for use in the composition include, but are not limited to polydimethylsiloxanes (PDMS) of high. molecular weight and gums of cellulose or of polysaccharides. Examples of pasty substances for use in the composition include, but are not limited to hydrocarbon compounds, such as lanolins and their derivatives, and PDMS.

As used herein, “pasty fat” is intended to mean a viscous product comprising a liquid fraction and a solid fraction. In one embodiment, at least one pasty fat for use in the composition has a melting point in the range from 20 to 55° C., for example from 25 to 45° C., and/or a viscosity at 40° C. in the range from 0.1 to 40 Pa·s (i.e., 1 to 400 poise), for example from 0.5 to 25 Pa·s. Viscosity may be measured with the Contraves TV or Rheomat 80. A person skilled in the art may choose the spindle for measuring the viscosity, from spindles MS-r3 and MS-r4, or on the basis of his general knowledge, so as to be able to carry out measurement of the viscosity of the pasty compound that is being tested.

The melting point values disclosed herein, unless otherwise noted, correspond to the melting peak measured by differential scanning calorimetry with a temperature rise of 5 or 10° C./min.

In at least one embodiment, said at least one fat is at least one hydrocarbon compound (comprising mainly carbon atoms and hydrogen and optionally ester groups), optionally of the polymeric type; said at least one hydrocarbon compound can also be chosen from silicone and/or fluorinated compounds; they may also be in the form of a mixture of at least one hydrocarbon and/or at least one silicone and/or at least one fluorinated compound. In the case of a mixture of different pasty fats, in one embodiment, at least one pasty hydrocarbon compound comprises the major fraction.

Examples of pasty compounds that can be used in the composition disclosed herein include, but are not limited to: the lanolins and lanolin derivatives; such as acetylated lanolins or oxypropylene lanolins or isopropyl lanolate; esters of acids or of aliphatic alcohols, for example those comprising 20 to 65 carbon atoms, such as tri-isostearyl or cetyl citrate; arachidyl propionate; vinyl polylaurate; cholesterol esters, such as triglycerides of vegetable origin, for example hydrogenated vegetable. oils, viscous polyesters such as poly(12-hydroxystearic) acid; and mixtures thereof. In one embodiment, at least one triglyceride of vegetable origin includes derivatives of hydrogenated castor oil.

Further examples of pasty compounds that can be used in the composition include pasty silicone, fats such as the polydimethylsiloxanes (PDMS) comprising pendent chains of the alkyl or alkoxy type comprising from 8 to 24 carbon atoms, such as the stearyl dimethicones.

The nature and amount of solids depend on the desired mechanical properties and textures. In one embodiment, the composition may contain an amount ranging from 0.1 to 50 wt. % of waxes relative to the total weight of the composition, for example from 1 to 30 wt. %.

The composition disclosed herein may additionally comprise, in a particular phase, at least one pigment and/or nacre and/or filler, such as those that are usually employed in cosmetic compositions.

The composition disclosed herein may also include at least one other coloring matter chosen from the water-soluble and/or fat-soluble colorants that are well known to a person skilled in the art.

As used herein, “pigments” are intended to mean particles of any shape, white or colored, mineral or organic, insoluble in the physiological medium, and intended to color the composition.

As used herein, “fillers” are intended to mean colorless or white particles, mineral and/or synthetic, lamellar and/or non-lamellar, intended to impart body and/or stiffness to the composition, and/or softness, malt appearance and/or evenness to make-up.

As used herein, “nacres” are intended to mean iridescent particles of any shape, such as iridescent particles produced in the shell of certain molluscs or such as iridescent particles that are synthesized.

In one embodiment, at least one pigment may be present in the composition in an amount ranging from 0.01 to 25 wt. % of the final composition, for example from 3 to 10 wt. %. In another embodiment, the at least one pigment may be white or colored, and in another embodiment it may be mineral or organic. Examples of pigments disclosed herein include, but are not limited to: the oxides of titanium, of zirconium or of cerium; the oxides of zinc, of iron or of chromium; ferric blue; chromium hydrate; carbon black; ultramarines (polysulphides of aluminosilicates); manganese pyrophosphate; and certain metal powders, such as those of silver or of aluminium. Further examples of pigments include D&C pigments and lakes that are commonly employed for imparting a make-up effect to the lips and to the skin, and which are salts of calcium, barium, aluminium, strontium or zirconium.

In one embodiment, at least one nacre may be present in the composition in an amount ranging from 0.01 to 20 wt. %, for example from 3 to 10 wt. %. Examples of nacres include, but are not limited to natural mother-of-pearl, mica coated with titanium dioxide or with iron oxide, with natural pigment or with bismuth oxychloride, and colored titanium mica.

In one embodiment, at least one fat-soluble or water-soluble dye may be present in the composition, alone or mixed, in an amount ranging from 0.001 to 15 wt. % relative to the total weight of the composition, for example from 0.01 to 5 wt. %, or from 0.1 to 2 wt. %. Examples of fat-soluble or water-soluble dyes include, but are not limited to: the disodium salt of ponceau; the disodium salt of alizarin green; quinoline yellow; the trisodium salt of amaranth; the disodium salt of tartrazine; the monosodium salt of rhodamine; the disodium salt of fuchsin; xanthophyll; methylene blue; carmine; halogen-acid dyes; azo dyes; anthraquinone dyes; copper or iron sulphate; Sudan brown; Sudan red; annatto; beetroot juice; and carotene.

In one embodiment, the composition disclosed herein may include at least one filler. In one embodiment, the at least one filler is present in an amount ranging from 0.01 to 50 wt. %, relative to the total weight of the composition, for example in the range from 0.02 to 30 wt. %. In one embodiment, the at least one filler may be mineral and/or organic, and of any shape, such as plate-like, spherical or oblong. Examples of fillers include, but are not limited to: talc; mica; silica; kaolin; powders of polyamide (Nylon®), of poly-β-alanine and of polyethylene; powders of tetrafluoroethylene (Teflon®) polymers; lauryl-lysine; starch; boron nitride; hollow polymeric microspheres, such as those of polyvinylidene chloride/acrylonitrile, for example Expancel® (Nobel Industrie); copolymers of acrylic acid (Polytrap® from the company Dow Corning) and microbeads of silicone resin (Tospearls® from Toshiba, for example); particles of elastomeric polyorganosiloxanes; precipitated calcium carbonate; magnesium carbonate and hydrocarbonate; hydroxyapatite; hollow silica microspheres (such as Silica Beads® from Maprecos); glass or ceramic microcapsules; metal soaps derived from organic carboxylic acids comprising from 8 to 22 carbon atoms, for example from 12 to 18 carbon atoms; zinc; magnesium or lithium stearate; zinc laurate; and magnesium myristate.

In one embodiment, the composition can further include at least one additional polymer, such as at least one film-forming polymer. As used herein, “film-forming polymer” is intended to mean a polymer that is able to form, on its own or in the presence of a film-forming additive, a continuous film that adheres to a substrate, for example to keratinous substances. Examples of film-forming polymers that can be used in the composition disclosed herein include, but are not limited to: synthetic polymers of the radical type or of the polycondensate type; polymers of natural origin; and mixtures thereof,

In one embodiment, at least one filler is chosen from acrylic polymers, polyurethanes, polyesters, polyamides, polyureas, and cellulosic polymers such as nitrocellulose.

In one embodiment, the composition disclosed herein can also include at least one ingredient that is commonly used in cosmetics, such as vitamins, thickeners, gelling agents, trace elements, softening agents, sequestering agents, perfumes, alkalizing or acidifying agents, preservatives, sun filters, surfactants, antioxidants, hair restorers, anti-dandruff agents, propellants, ceramides, film-forming additives, and/or mixtures thereof.

Of course, a person skilled in the art will take care to select any additional compound or compounds, and/or their amount, in such a way that the advantageous properties of the composition disclosed herein are not, or substantially not, adversely affected by the addition envisioned.

In one embodiment, the composition disclosed herein may be in the form of a suspension. Examples of such suspensions include, but are not limited to: a dispersion of oil in water owing to the presence of vesicles; an oily solution optionally thickened or gelled; an oil-in-water, water-in-oil, or multiple emulsion; a gel or a mousse; an oily or emulsified gel; a dispersion of vesicles, such as lipid vesicles; a two-phase or multiphase lotion; a spray; a free, compact or cast powder; and an anhydrous paste. In one embodiment, said composition may have the appearance of a lotion, a cream, an ointment, a soft paste, an unguent, a mousse, a cast or a molded solid. In a further embodiment, said composition is in the form of a stick, or in a dish, or in the form of a compacted solid.

A person skilled in the art will be able to select the appropriate galenical form, as well as its method of preparation, on the basis of his general knowledge, taking into account on the one hand the nature of the constituents used, for example their solubility in the carrier, and on the other hand the application envisioned for the composition.

In one embodiment, the cosmetic composition disclosed herein is in the form of a product for care and/or make-up of the skin of the body or of the face, the lips, the nails, the eyelashes, the eyebrows and/or the hair, a sun-tan or self-tanning product, or a hair product for the care, treatment, shaping, make-up or coloring of the hair.

In another embodiment, the composition can thus be in the form of a make-up composition, such as a product for the complexion such, as a foundation, a blusher or eye shadow; a product for the lips such as a lipstick or for care of the lips; a concealer; a blusher, a mascara, an eye-liner; a product for make-up of the eyebrows, a pencil for the lips or the eyes; a product for the nails such as a nail varnish or for care of the nails; a product for make-up of the body; a product for make-up of the hair (mascara or hair lacquer).

In another embodiment, the composition is in the form of a composition for protection or for care of the skin of the face, neck, hands or body, such as an anti-wrinkle composition or a moisturizing or medicated composition; or an anti-sun or artificial tanning composition.

In another embodiment, the composition may be in the form of a hair-care product, such as a product for coloring, hairstyle form retention, hair shaping, care, treatment or cleaning of the hair, such as shampoos, gels, setting lotions, lotions for blow drying, and compositions for fixation and styling such as lacquers or sprays.

In one embodiment, the cosmetic composition disclosed herein is in the form of a make-up product, for example a foundation, or of a care product such as a cream for care of the face or a sun-tan product.

Also disclosed herein is a method of cosmetic treatment, for example for make-up or care of keratinous substances, such as the skin of the body or of the face, the lips, the nails, the eyelashes, the eyebrows and/or the hair, comprising the application, on said substances, of a cosmetic composition as defined previously.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

Shine Measured with the Shine-o-Meter on a Dry Deposit of Polymer

Shine can be measured using a shine-o-meter, conventionally by the following method.

On a LENETA contrast card of reference FORM 1A PENOPAC, a 50 μm thick layer of a solution of the polymer at 50% in isododecane is spread using an automatic spreader. The layer covers at least the dark background of the card. The deposit is left to dry for 24 hours at a temperature of 25° C., then the shine is measured at 200 on the dark background using a shine-o-meter of the DR LANGE type, REF03. The shine is also measured at 60°, then at 85°.

A measurement at 20° below about 50 gives a matt appearance that is deemed acceptable, and very satisfactory if the measurement result is below 40.

### Oil Resistance of the Polymers by Measurement of Stickiness

This is determined with a drop of olive oil placed on a dry polymer film.

A polymer film is prepared from a 20% solution of polymer in isododecane; 0.5 ml is spread on a glass plate with dimensions of 2.5×7.5 cm and left to dry at room temperature (25° C.) for 24 hours. Then 1 ml of olive oil is spread on the polymer film.

After 1 hour, the excess oil is wiped from the film and the stickiness is assessed by the touch, by comparing with the comparative polymer.

A mark of ‘***’ or ‘3*’ is given when the plate is lifted off with the finger (very sticky).

A mark of ‘0’ signifies: no stickiness detected.

The stickiness reflects the sensitivity of the polymer to olive oil. The stickier the polymer is in the presence of oil, the more sensitive it is to the oil and therefore the deposit will be affected more easily, for example during a meal (in the presence of oil in foods) or by sebum. As a result, the polymer is retained less well on the skin. This also leads to a decrease in comfort: the stickier the film, the more uncomfortable the composition is when it is being worn.

### Internal Stresses

A polymer film is prepared from a 20% solution of polymer in isododecane; 30 microliters is spread on a strip of nitrile of dimensions 6×1 cm and it is left to dry at room temperature (25° C.) for 3 hours. The deformation of the substrate after drying is examined visually.

A mark of 0 signifies that the substrate did not deform during drying.

A mark of +++ signifies that the substrate is almost folded (completely deformed) after drying.

The internal stresses (ISs) reflect comfort from the mechanical standpoint: when there is no deformation of the substrate during drying, it may be thought that there will be no discomfort during drying of the composition after its application on the skin.

### EXAMPLE 1

30 g of isododecane was placed in a 250-ml three-necked flask, then heated to 90° C. In the space of 30 minutes, 4.5 g of isobornyl acrylate, 4.5 g of isobornyl methacrylate and 0.37 g of initiator 2,5-bis(2-ethylhexanoylperoxy)-2,5-dimethylhexane (Trigonox® 141 from Akzo Nobel) were added. The whole was maintained at 90° C. for 1.5 h.

Then, to the aforesaid mixture, at 90° C. and in 30 minutes, 21 g of MPTS and 0.29 g of 2,5-bis(2-ethylhexanoylperoxy)-2,5-dimethylhexane were added. The mixture was held at 90° C. for 3 hours, then the Whole was cooled to 25° C.

A 51.5% solution of polymer dry matter in isododecane was obtained. Said polymer comprised a first block comprising isobornyl (meth)acrylates and a second block comprising the silicone monomer.

### EXAMPLE 2

30 g of isododecane was placed in a 250-ml three-necked flask, then heated to 90° C. In the space of 30 minutes, 7.5 g of isobornyl acrylate, 7.5 g of isobornyl methacrylate and 0.30 g of initiator 2,5-bis(2-ethylhexanoylperoxy)-2,5-dimethylhexane (Trigonox® 141 from Akzo Nobel) were added. The whole was maintained at 90° C. for 1.5 h.

Then, to the aforesaid mixture, at 90° C. and in 30 minutes, 15.2 g of MPTS and 0.20 g of 2,5-bis(2-ethylhexanoylperoxy)-2,5-dimethylhexane were added. The mixture was held at 90° C. for 3 hours, then the whole was cooled to 25° C.

A 51.2% solution of polymer dry matter in isododecane was obtained. Said polymer comprised a first block comprising isobornyl (meth)acrylates and a second block comprising the silicone monomer.

### EXAMPLE 3

60 g of isododecane was placed in a 250-ml three-necked flask, then heated to 90° C. In the space of 30 minutes, 21 g of isobornyl acrylate, 21 g of isobornyl methacrylate and 0.36 g of initiator 2,5-bis(2-ethylhexanoylperoxy)-2,5-dimethylhexane (Trigonox® 141 from Akzo Nobel) were added. The whole was maintained at 90° C. for 2.5 h.

Then, to the aforesaid mixture, at 90° C. and in 30 minutes, 18 g of MPTS and 0.25 g of 2,5-bis(2-ethylhexanoylperoxy)-2,5-dimethylhexane were added. The mixture was held at 90° C. for 3 hours, then the whole was cooled to 25° C.

A 53.1% solution of polymer dry matter in isododecane was obtained. Said polymer comprised a first block comprising isobornyl (meth)acrylates and a second block comprising the silicone monomer.

### EXAMPLE 4

30 g of isododecane was placed in a 250-ml three-necked flask, then heated to 90° C. In the space of 30 minutes, 21 g of isobornyl methacrylate and 0.19 g of initiator 2,5-bis(2-ethylhexanoylperoxy)-2,5-dimethylhexane (Trigonox® 141 from Akzo Nobel) were added. The whole was maintained at 90° C. for 1.5 h.

Then, to the aforesaid mixture, at 90° C. and in 30 minutes, 9 g of MPTS and 0.12 g of 2,5-bis(2-ethylhexanoylperoxy)-2,5-dimethylhexane were added. The mixture was held at 90° C. for 3 hours, then the whole was cooled to 25° C.

A 52.8% solution of polymer dry matter in isododecane was obtained. Said polymer comprised a first block comprising isobornyl methacrylate and a second block comprising the silicone monomer.

### EXAMPLE 5

30 g of isododecane was placed in a 250-ml three-necked flask, then heated to 90° C. In the space of 30 minutes, 15 g of isobornyl methacrylate and 0.30 g of initiator 2,5-bis(2-ethylhexanoylperoxy)-2,5-dimethylhexane (Trigonox® 141 from Akzo Nobel) were added. The whole was maintained at 90° C. for 1.5 h.

Then, to the aforesaid mixture, at 90° C. and in 30 minutes, 15 g of MPTS and 0.20 g of 2,5-bis(2-ethylhexanoylperoxy)-2,5-dimethylhexane were added. The mixture was held at 90° C. for 3 hours, then the whole was cooled to 25° C.

A 51.7% solution of polymer dry matter in isododecane was obtained. Said polymer comprised a first block comprising isobornyl methacrylate and a second block comprising the silicone monomer.

### EXAMPLE 6

30 g of isododecane was placed in a 250-ml three-necked flask, then heated to 90° C. In the space of 30 minutes, 7.5 g of isobornyl acrylate, 7.5 g of isobornyl methacrylate and 0.33 g of initiator tert-butyl peroxy-2-ethylhexanoate (Trigonox® 21S from Akzo Nobel) were added. The whole was maintained at 90° C. for 1.5 h.

Then, to the aforesaid mixture, at 90° C. and in 30 minutes, 13.5 g of MPTS, 1.5 g of acrylic acid and 0.22 g of Trigonox 21S were added. The mixture was held at 90° C. for 3 hours, then the whole was cooled to 25° C.

A 56.2% solution of polymer dry matter in isododecane was obtained. Said polymer comprised a first block comprising isobornyl (meth)acrylates and a second block comprising the silicone monomer and acrylic acid.

### EXAMPLE 7

30 g of isododecane was placed in a 250-ml three-necked flask, then heated to 90° C. In the space of 30 minutes, 4.5 g of isobornyl acrylate, 4.5 g of isobornyl methacrylate and 0.37 g of initiator 2,5-bis(2-ethylhexanoylperoxy)-2,5-dimethylhexane (Trigonox® 141 from Akzo Nobel) were added. The whole was maintained at 90° C. for 1.5 h.

Then, to the aforesaid mixture, at 90° C. and in 30 minutes, 19.5 g of MPTS, 1.5 g of acrylic acid and 0.29 g of 2,5-bis(2-ethylhexanoylperoxy)-2,5-dimethylhexane were added. The mixture was held at 90° C. for 3 hours, then the whole was cooled to 25° C.

A 55.4% solution of polymer dry matter in isododecane was obtained. Said polymer comprised a first block comprising isobornyl (meth)acrylates and a second block comprising the silicone monomer and acrylic acid.

### EXAMPLE 8

30 g of isododecane was placed in a 250-ml three-necked flask, then heated to 90° C. In the space of 30 minutes, 9 g of isobornyl methacrylate and 0.37 g of initiator 2,5-bis(2-ethylhexanoylperoxy)-2,5-dimethylhexane (Trigonox® 141 from Akzo Nobel) were added. The whole was maintained at 90° C. for 1.5 h.

Then to the aforesaid mixture, at 90° C. and in 30 minutes, 21 g of MPTS and 0.28 g of 2,5-bis(2-ethylhexanoylperoxy)-2,5-dimethylhexane were added. The mixture was held at 90° C. for 3 hours, then the whole was cooled to 25° C.

A 52.3% solution of polymer dry matter in isododecane was obtained. Said polymer comprised a first block comprising isobornyl methacrylate and a second block comprising the silicone monomer.

### EXAMPLE 9

30 g of isododecane was placed in a 250-ml three-necked flask, then heated to 90° C. In the space of 1 hour, 10.5 g of isobornyl acrylate, 10.5 g of isobornyl methacrylate and 0.19 g of initiator tert-butyl peroxy-2-ethylhexanoate (Trigonox® 21S from Akzo Nobel) were added. The whole was maintained at 90° C. for 1.5 h.

Then to the aforesaid mixture, at 90° C. and in 30 minutes, 8.25 g of MPTS, 0.75 g of acrylic acid and 0.13 g of Trigonox 21S were added. The mixture was held at 90° C. for 3 hours, then the whole was cooled to 25° C.

A 54.6% solution of polymer dry matter in isododecane was obtained. Said polymer comprised a first block comprising isobornyl (meth)acrylates and a second block comprising the silicone monomer and acrylic acid.

### COMPARATIVE EXAMPLE

### Not Part of the Invention

100 g of isododecane was placed in a 1-liter reactor, then the temperature was raised so that it rose from room temperature (25° C.) to 90° C. in one hour.

Then at 90° C. and in 1 hour, 105 g of isobornyl acrylate, 105 g of isobornyl methacrylate, 110 g of isododecane and 1.8 g of 2,5-bis(2-ethylhexanoylperoxy)-2,5-dimethylhexane (Trigonox® 141 from Akzo Nobel) were added. The mixture was held at 90° C. for 1.5 h.

Then, to the aforesaid mixture, still at 90° C. and in 30 minutes, 90 g of isobutyl acrylate, 90 g of isododecane and 1.2 g of 2,5-bis(2-ethylhexanoylperoxy)-2,5-dimethylhexane were added. The mixture was held at 90° C. for 3 hours, then the whole was cooled.

A 50% solution of polymer active substance in isododecane was obtained. Said polymer comprised a first block comprising isobornyl (meth)acrylates and a second block comprising isobutyl acrylate.

### EXAMPLE 10

The films obtained with the polymers of the present disclosure from examples 1 to 9, as well as with the comparative polymer prepared above, were assessed.

Film: stickiness, the shine and the friability of a film only of polymer were determined by visual examination.

The shine, the stickiness (resistance to olive oil) and the internal stresses of the polymer were determined according to the methods stated above.

The following results were obtained:

TABLE 1

| | Example 1 | Example 2 | Example 3 | Example 4 | Example 5 |
|---|---|---|---|---|---|
| % A. Isob. | 15 | 25 | 35 | — | — |
| % M. Isob. | 15 | 25 | 35 | 70 | 50 |
| % MPTS | 70 | 50 | 30 | 30 | 50 |
| Mw (g/mol) | 67,700 | 61,900 | 67,800 | 95,400 | 68,400 |
| Ip | 7.85 | 5.99 | 4.49 | 3.83 | 2.82 |
| DE (%) | 51.5% | 51.2% | 53.1% | 52.8% | 51.7% |
| Film only | a bit shiny | matt | white | matt | matt |
| (visual examination) | a bit sticky | not sticky | not sticky | not sticky | not sticky |
| | not brittle | not brittle | very slightly | slightly | very slightly |
| | | | brittle | brittle | brittle |
| Shine | 20°: 46 | 20°: 1 | 20°: 1.6 | 20°: 1.2 | 20°: 1.2 |
| | 60°: 76 | 60°: 4.4 | 60°: 7.5 | 60°: 2.7 | 60°: 3.3 |
| | 85°: 93.4 | 85°: 13.2 | 85°: 23.8 | 85°: 3.7 | 85°: 6.6 |
| Stickiness | ++ | + | + | 0 | + |
| Internal | 0 | 0 | ++ | + | 0 |
| stresses | | | | | |

TABLE 2

| | Example 6 | Example 7 | Example 8 | Example 9 | Comparative |
|---|---|---|---|---|---|
| % A. Isob. | 25 | 15 | — | 35 | 35 |
| % M. Isob. | 25 | 15 | 30 | 35 | 35 |
| % MPTS | 45 | 65 | 70 | 27.5 | — |
| % AA | 5 | 5 | — | 2.5 | — |
| % A. Ibut. | — | — | — | — | 30 |
| Mw (g/mol) | 168,600 | 663,600 | 86,500 | 104,000 | 100,300 |
| Ip | 8.22 | 32.75 | 3.69 | 4.52 | 4.40 |
| DE (%) | 56.2% | 55.4% | 52.3% | 54.6% | 50% |
| Film only | matt, slightly | opaque, | matt | matt | shiny |
| (visual | opaque | slightly shiny | | | |
| examination) | not sticky | not sticky | a bit sticky | not sticky | not sticky |
| | not brittle | very slightly | not brittle | not brittle | brittle |
| | | brittle | | | |
| Shine | 20°: 2.4 | 20°: 44.1 | 20°: 8.5 | 20°: 5.2 | 20°: 71.3 |
| | 60°: 15.2 | 60°: 75.1 | 60°: 37 | 60°: 26.2 | 60°: 86.6 |
| | 85°: 23.2 | 85°: 95.5 | 85°: 62.9 | 85°: 35.9 | |
| Stickiness | 0 | + | ++ | 0 | +++ |
| Internal | + | 0 | 0 | + | +++ |
| stresses | | | | | |

As used in Tables 1 and 2, “A. Isob.” is isobornyl acrylate, “M. Isob.” is isobornyl methacrylate, “MPTS” is methacryloxypropyltris(trimethylsiloxy)silane, “AA” is acrylic acid, “A. Ibut” is isobutyl acrylate, and “DE” is dry extract.

### EXAMPLE 11

A foundation was prepared as follows:

the pigments (phase A3) were ground 3 times in a three-roller mill in cyclopentasiloxane;

the constituents of phase A1 were mixed in a Moritz mixer at room temperature, then phases A2, A3, A4 and A5 were added, taking care to homogenize well between each phase and to increase the stirring speed if necessary;

then phase B was added progressively, increasing the stirring speed if necessary, and stirring was continued for 10 minutes.

TABLE 3

| Phase | Proportion (%) | Compound |
|---|---|---|
| A1 | 1.8 | Bis PEG/PPG-14/14 dimethicone |
| | 0.6 | Isostearyl diglyceryl succinate |
| | Qsf 100% | Isododecane |
| | 5.0 | Cyclopentasiloxane (D5) |
| A2 | 4.25 | Polymer from ex. 7 (i.e. 2.2% MS of polymer) |
| A3 | 5.0 | Cyclopentasiloxane (D5) |
| | 3.5 | Coated iron oxides |
| | 6.8 | Coated titanium dioxide |
| A4 | 3.0 | Silicone gum in D5 |
| A5 | 8.0 | Nylon 12 |
| B | 40.0 | Water |
| | Qs | Preservative |
| | 0.7 | Magnesium sulphate |

This foundation composition, applied to the face, provided satisfactory properties of comfort and matt appearance.

What is claimed is:

1. A cosmetic and/or pharmaceutical composition comprising, in at least one physiologically acceptable medium, at least one block (co)polymer comprising at least one first block and at least one second block, wherein at least one of the blocks comprises at least one silicone monomer present in an amount ranging from 35 to 100 wt. %, relative to the weight of said block, and wherein the at least one silicone monomer is chosen from:
(i) ethylenic monomers comprising at least one ester group which comprises silanes and/or siloxanes, of formula: 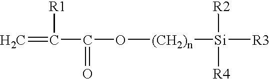
wherein:
R1 is chosen from H and methyl;
R2, R3, R4, which may be identical or different, comprise from C₁ to C₆ alkyl groups or —OSi(R5)₃ groups, wherein R5 is chosen from methyl and ethyl; and
n is an integer ranging from 1 to 10;
(ii) PDMS macromonomers and polydimethylsiloxanes comprising at least one monoacryloyloxy and/or monomethacryloyloxy end group, of the following formula: 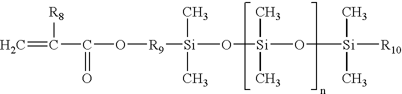
wherein:
R8 is chosen from hydrogen atoms and methyl groups;
R9 is chosen from linear or branched divalent hydrocarbon groups, comprising from 1 to 10 carbon atoms, and optionally comprising one or two ether bonds —O—;
R10 is chosen from linear or branched alkyl groups, comprising from 1 to 10 carbon atoms; and
n is an integer ranging from 1 to 300.
(iii) ethylenic monomers comprising at least one ester group which comprises at least one dendrimeric carboxysilane of formula: 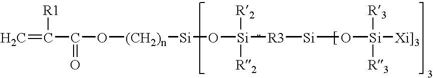
wherein:
R1 is chosen from H and methyl;
n is an integer ranging from 1 to 10;
R′2, R″2, R′3 and R″3, which may be identical or different, comprise at least one from C₁ to C₁₀ alkyl group;
R3 comprises at least one from C₂ to C₁₀ divalent alkylene group;
i is an integer ranging from 1 to 10; and
when i is an integer ranging from 2 to 10, X(i), which may be identical or different, comprises at least one —R4-Si—[O—(R′3)(R″3)-X(i−1)]₃ group, wherein R4, which may be identical or different comprises at least one from C₂ to C₁₀ divalent alkylene group;
and when i is 1, X(1) is chosen from H and from C₁ to C₁₀ alkyl groups;
(iv) ethylenic monomers of type POSS and POS of structure: 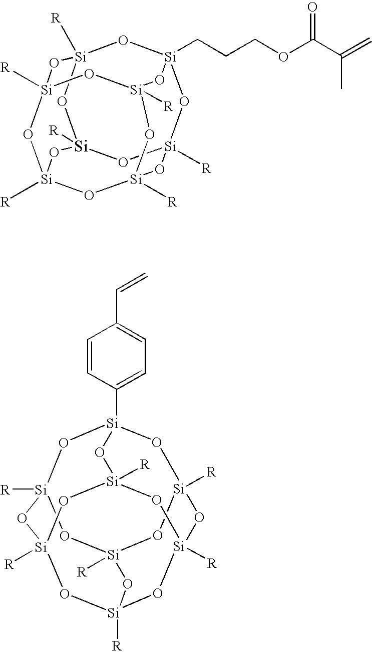
wherein R, which may be identical or different, is chosen from C₁ to C₁₀ linear alkyl groups and C₃ to C₁₂ cyclic alkyl groups;
(v) and mixtures thereof.

2. The composition according to claim 1, wherein the at least one silicone monomer is chosen from:
(meth)acryloxypropyltris(trimethylsiloxy)silane and (meth)acryloxypropyl-bis(trimethylsiloxy)methylsilane;
(meth)acryloxymethyltris(trimethylsiloxy)silane and (meth)acryloxymethyl-bis(trimethylsiloxy)methylsilane;
(meth)acryloxypropyltrimethoxysilane;
the following monomers, wherein R1 is chosen from H and methyl: 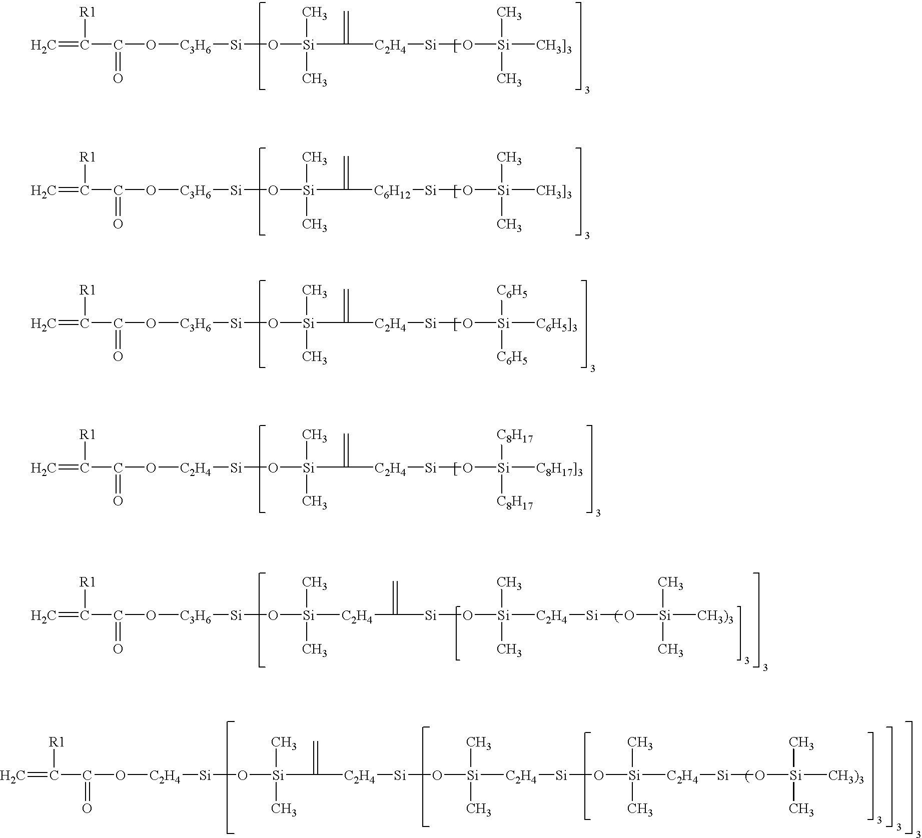
and mixtures thereof.

3. The composition according to claim 1, wherein the at least one silicone monomer is present in an amount ranging from 50 to 99 wt. % relative to the total weight of the block containing the at least one silicone monomer.

4. The composition according to claim 1, wherein the at least one silicone monomer is present in an amount ranging from 20 to 90 wt. %, relative to the total weight of the polymer.

5. The composition according to claim 1, wherein the polymer additionally comprises at least one carbon-containing monomer, wherein said at least one carbon containing monomer is present in an amount ranging from 10 to 80 wt. % relative to the total weight of the composition.

6. The composition according to claim 5, wherein the at least one carbon-containing monomer is chosen from the following monomers:
methacrylates of formula CH₂═C(CH₃)—COOR1 wherein R1 is chosen from: linear or branched C₈ to C₂₂ alkyl groups; cyclic alkyl groups comprising from 8 to 30 carbon atoms; and tert-butyl groups;
acrylates of formula CH₂═CH—COOR2 wherein R2 is chosen from: linear or branched C₈ to C₂₂ alkyl groups; cyclic alkyl groups comprising from 8 to 30 carbon atoms; and isobutyl groups;
(meth)acrylamides of formulas CH₂═C(CH₃)—CONR3R4 and CH₂═CH—CONR3R4, wherein R3 is chosen from hydrogen atoms and linear or branched C₁ to C₁₂ alkyl groups, and wherein R4 is chosen from linear or branched C₈ to C₁₂ alkyl groups;
di-n-alkyl itaconates of formula CH₂═C(CH₂—COO(CH₂)ₙ.CH₃)COO(CH₂)ₙ.CH₃, wherein n is an integer greater than or equal to 5;
vinyl esters of formula CH₂═CHOCOR5 wherein R5 is chosen from linear or branched C₈ to C₂₂ alkyl groups;
vinyl ethers of formula CH₂═CH—OR6 wherein R6 is chosen from linear or branched alkyl groups, comprising from 8 to 22 carbon atoms;
linear and branched C₆ to C₂₂ alkyl (meth)acrylate homopolymers and copolymers, comprising at least one polymerizable end group chosen from the vinyl or (meth)acrylate groups;
polyolefins comprising at least one end group comprising at least one ethylenic unsaturation; and
mixtures thereof.

7. The composition according to claim 5, wherein the at least one carbon-containing monomer is chosen from: lauryl, behenyl, stearyl, isobornyl, and tert-butyl methacrylates; lauryl, behenyl, 2-ethylhexyl, stearyl, isobornyl, and isobutyl acrylates; macromonomers comprising poly(ethyl-2-hexyl acrylate) and/or poly(dodecyl acrylate) comprising at least one mono(meth)acrylate end group; ethylene/butylene and ethylene/propylene macromonomers comprising at least one (meth)acrylate reactive end group; and mixtures thereof.

8. The composition according to one claim 1, wherein the polymer additionally comprises at least one additional monomer chosen from the following monomers:
(i) ethylenic hydrocarbons comprising from 2 to 10 carbons;
(ii) (meth)acrylates of formulas CH₂═CHCOOR′₃ and CH₂═C(CH₃)COOR′₃ wherein R′₃ is chosen from:
linear or branched alkyl groups comprising from 1 to 7 carbon atoms, wherein said alkyl groups are optionally interrupted by at least one heteroatom chosen from O, N, S and P; and optionally substituted with at least one substituent chosen from hydroxyl groups and halogen atoms;
C₃ to C₇ cycloalkyl groups;
C₃ to C₂₀ aryl groups;
C₄ to C₃₀ aralkyl groups (C₁ to C₈ alkyl group);
heterocyclic groups comprising from 4 to 12 ring members comprising at least one heteroatom chosen from O, N, and S, wherein the ring is aromatic or not aromatic;
heterocycloalkyl (C₁ to C₄ alkyl) groups,
wherein said cycloalkyl, aryl, aralkyl, heterocyclic or heterocycloalkyl groups are optionally substituted with at least one substituent chosen from hydroxyl groups, halogen atoms, and linear and branched C₁₋₄ alkyl groups, wherein said alkyl groups are optionally interrupted by at least one heteroatom chosen from O, N, S and P, and optionally substituted with at least one substituent chosen from hydroxyl groups and halogen atoms,
—(C₂H₄O)ₘ—R″ groups, wherein m is an integer ranging from 5 to 150 and R″ is chosen from H and from C₁ to C₃₀ alkyl groups; and
isobutyl methacrylate and tert-butyl acrylate;
(iii) (meth)acrylamides of formula: 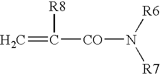
wherein R₈ is chosen from H and methyl; and wherein R₇ and R₆, which may be identical or different, are chosen from:
hydrogen atoms;
linear and branched alkyl groups comprising from 1 to 22 carbon atoms, said alkyl groups being optionally interrupted by at least one heteroatom chosen from O, N, S and P, and optionally substituted by at least one substituent chosen from hydroxyl groups and halogen atoms;
C₃ to C₁₂ cycloalkyl groups;
C₃ to C₂₀ aryl groups;
C₄ to C₃₀ aralkyl groups (C₁ to C₈ alkyl group);
heterocyclic groups comprising from 4 to 12 ring members comprising at least one heteroatom chosen from O, N, and S, wherein the ring is aromatic or not aromatic; and
heterocycloalkyl (C₁ to C₄ alkyl) groups;
wherein said cycloalkyl, aryl, aralkyl, heterocyclic and heterocycloalkyl groups are optionally substituted with at least one substituent chosen from hydroxyl groups, halogen atoms, and linear or branched C₁-C₄ alkyl groups, said alkyl groups being optionally substituted by at least one heteroatom chosen from O, N, S and P, and optionally substituted with at least one substituent chosen from hydroxyl groups and halogen atoms;
(iv) vinylic compounds of formulas:
CH₂═CH—R₉, CH₂═CH—CH₂—R₉ and CH₂═C(CH₃)—CH₂—R₉
wherein R₉ is chosen from: hydroxyl groups, halogens, NH₂, and OR₁₄ wherein R₁₄ comprises at least one phenyl group or at least one from C₁ to C₇ alkyl group; acetamide (NHCOCH₃), and OCOR₁₅ groups wherein R₁₅ is chosen from linear or branched alkyl groups comprising from 2 to 7 carbon atoms, and groups chosen from:
linear or branched alkyl groups, comprising from 1 to 22 carbon atoms, wherein said alkyl groups are optionally interrupted by at least one heteroatom chosen from O, N, S and P; and optionally substituted with at least one substituent chosen from hydroxyl groups and halogen atoms;
C₃ to C₁₂ cycloalkyl groups;
C₃ to C₂₀ aryl groups;
C₄ to C₃₀ aralkyl groups (C₁ to C₈ alkyl group);
heterocyclic groups comprising from 4 to 12 ring members comprising at least one heteroatom chosen from O, N, and S, wherein the ring is aromatic or not aromatic,
heterocycloalkyl (C₁ to C₄ alkyl) groups,
wherein said cycloalkyl, aryl, aralkyl, heterocyclic or heterocycloalkyl groups are optionally substituted with at least one substituent chosen from hydroxyl groups, halogen atoms, and linear and branched alkyl groups comprising 1 to 4 carbon atoms, said alkyl groups being optionally interrupted by at least one heteroatom chosen from O, N, S and P, and optionally substituted with at least one substituent chosen from hydroxyl groups and halogen atoms;
(v) monomers comprising at least one ethylenic unsaturation comprising at least one carboxylic, phosphoric or sulphonic acid function, or anhydride, and salts thereof;
(vi) monomers comprising at least one ethylenic unsaturation which comprises at least one tertiary amine function;
(vii) and mixtures thereof.

9. The composition according to claim 8, wherein the at least one additional monomer is chosen from: (meth)acrylic acid and (meth)acrylates of C₁ to C₄ alkyl, or of C₃ to C₇ cycloalkyl; isobutyl methacrylate; tert-butyl acrylates; acrylic acid and methacrylic acid; and mixtures thereof.

10. The composition according to claim 1, wherein said at least one first and at least one second blocks are linked together by at least one intermediate block or sequence comprising at least one monomer constituting said at least one first block and at least one monomer constituting said at least one second block.

11. The composition according to claim 1, wherein the polymer is present in an amount ranging from 0.01 to 50 wt. % relative to the total weight of the composition.

12. The composition according to claim 1, wherein the at least one physiologically acceptable medium comprises at least one constituent chosen from: water, hydrophilic organic solvents, waxes, pasty fats, gums, and mixtures thereof; lipophilic organic solvents; oils; pigments, nacres, fillers, water-soluble colorants, and fat-soluble colorants; polymers; vitamins, thickeners, gelling agents, trace elements, softening agents, sequestering agents, perfumes, alkalizing or acidifying agents, preservatives, sun filters, surfactants, antioxidants, hair restorers, anti-dandruff agents, propellants, ceramides, film-forming additives, and mixtures thereof.

13. The composition according to claim 1, wherein said composition is in the form of a product for care and/or make-up of the skin of the body or face, the lips, the nails, the eyelashes, the eyebrows and/or the hair, a sun-tan and/or self-tanning product, and/or a hair product for the care, treatment, shaping, make-up and/or coloring of the hair.

14. A method of cosmetic. treatment for make-up and/or care of keratinous substances, comprising applying to said keratinous substances a cosmetic composition comprising, in at least one physiologically acceptable medium, at least one block (co)polymer comprising at least one first block and at least one second block, wherein at least one of the blocks comprises at least one silicone monomer present in an amount ranging from 35 to 100 wt. %, relative to the weight of said block, and wherein the at least one silicone monomer is chosen from:
(i) ethylenic monomers comprising at least one ester group which comprises silanes and/or siloxanes, of formula: 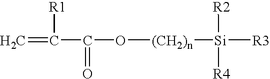
wherein:
R1 is chosen from H and methyl;
R2, R3, R4, which may be identical or different, comprise C₁ to C₆ alkyl groups or —OSi(R5)₃ groups, wherein R5 is chosen from methyl and ethyl; and
n is an integer ranging from 1 to 10;
(ii) PDMS macromonomers and polydimethylsiloxanes comprising at least one monoacryloyloxy and/or monomethacryloyloxy end group, of the following formula: 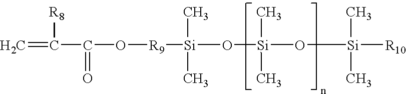
wherein:
R8 is chosen from hydrogen atoms and methyl groups;
R9 is chosen from linear or branched divalent hydrocarbon groups, comprising from 1 to 10 carbon atoms, and optionally comprising one or two ether bonds —O—;
R10 is chosen from linear or branched alkyl groups, comprising from 1 to 10 carbon atoms; and
n is an integer ranging from 1 to 300.
(iii) ethylenic monomers comprising at least one ester group which comprises at least one dendrimeric carboxysilane of formula: 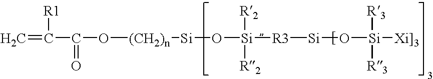
wherein:
R1 is chosen from H and methyl;
n is an integer ranging from 1 to 10;
R′2, R″2, R′3 and R″3, which may be identical or different, comprise at least one from C₁ to C₁₀ alkyl group;
R3 comprises at least one from C₂ to C₁₀ divalent alkylene group;
i is an integer ranging from 1 to 10; and
when i is an integer ranging from 2 to 10, X(i), which may be identical or different, comprises at least one —R4-Si—[O—(R′3)(R″3)-X(i−1)]₃ group, wherein R4, which may be identical or different comprises at least one from C₂ to C₁₀ divalent alkylene group;
and when i is 1, X(1) is chosen from H and from C₁ to C₁₀ alkyl groups;
(iv) ethylenic monomers of type POSS and POS of structure: 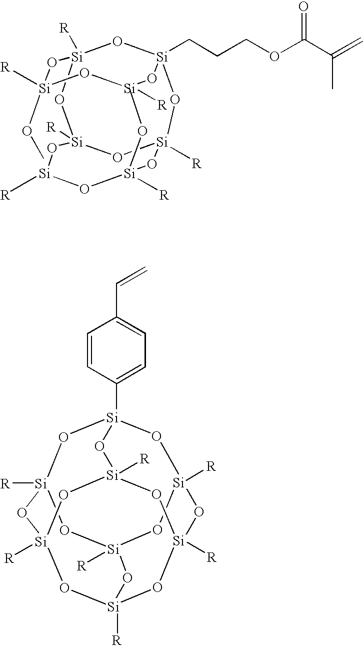
wherein R, which may be identical or different, is chosen from C₁ to C₁₀ linear alkyl groups and C₃ to C₁₂ cyclic alkyl groups;
(v) and mixtures thereof.

15. A block (co)polymer comprising at least one first block and at least one second block, wherein at least one block comprises at least one silicone monomer in an amount ranging from 35 to 100 wt. % relative to the total weight of said block, wherein said at least one silicone monomer is chosen from:
(i) ethylenic monomers comprising at least one ester group which comprises at least one silane and/or siloxane, of formula: 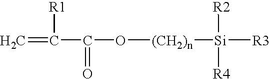
wherein:
R1 is chosen from H and methyl;
R2, R3, R4, which may be identical or different, comprise C₁ to C₆ alkyl groups and/or —OSi(R5)₃ groups, wherein R5 is chosen from methyl and ethyl; and
n is an integer ranging from 1 to 10;
(ii) tPDMS macromonomers and polydimethylsiloxanes comprising at least one monoacryloyloxy and/or monomethacryloyloxy end group of the following formula 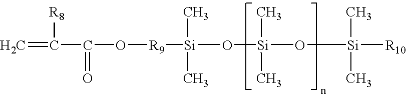
wherein:
R8 is chosen from hydrogen atoms and methyl groups;
R9 is chosen from linear or branched divalent hydrocarbon groups comprising from 1 to 10 carbon atoms and optionally comprising one or two ether bonds —O—;
R10 is chosen from a linear and branched alkyl groups, comprising from 1 to 10 carbon atoms; and
n is an integer ranging from 1 to 300.
(iii) ethylenic monomers comprising at least one ester group which comprises at least one dendrimeric carboxysilane of formula: 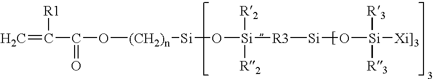
wherein:
R1 is chosen from H and methyl;
n is an integer ranging from 1 to 10;
R′2, R″2, R′3 and R″3, which may be identical or different, comprise at least one from C₁ to C₁₀ alkyl group;
R3 comprises at least one from C₂ to C₁₀ divalent alkylene group;
i is an integer ranging from 1 to 10; and
when i is an integer ranging from 2 to 10, X(i), which may be identical or different, comprises at least one —R4-Si—[O—(R′3)(R″3)-X(i−1)]₃ group, wherein R4, which may be identical or different, comprises at least one from C₂ to C₁₀ divalent alkylene group; and
when i is 1, X(1) is chosen from H and from C₁ to C₁₀ alkyl groups;
(iv) ethylenic monomers of type POSS and POS of structure: 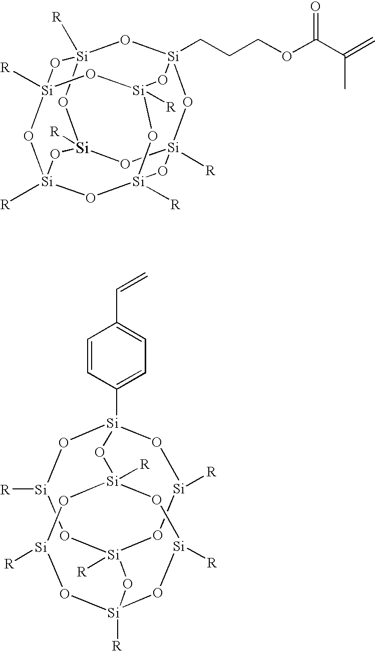
wherein R, which may be identical or different, is chosen from C₁ to C₁₀ linear and from C₃ to C₁₂ cyclic alkyl groups; and
(v) mixtures thereof.

16. The polymer according to claim 15, wherein the at least one silicone monomer is chosen from:
(meth)acryloxypropyltris(trimethylsiloxy)silane, and (meth)acryloxypropyl-bis(trimethylsiloxy)methylsilane;
(meth)acryloxymethyltris(trimethylsiloxy)silane, and (meth)acryloxymethyl-bis(trimethylsiloxy)methylsilane;
(meth)acryloxypropyltrimethoxysilane;
the following monomers, wherein R1 is chosen from H and methyl: 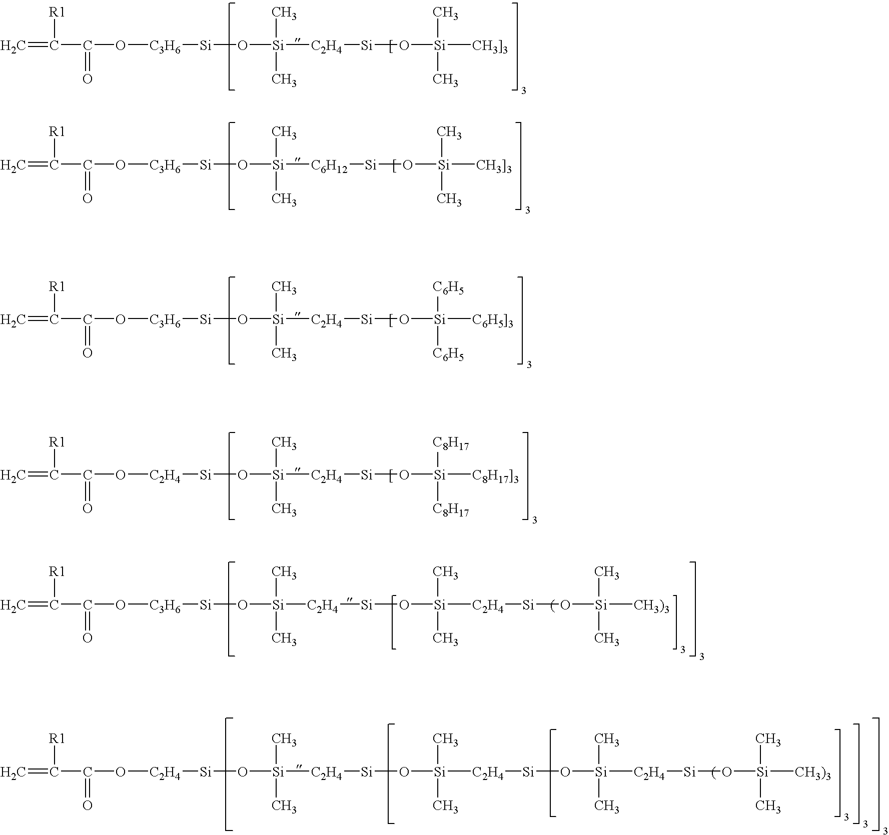
and mixtures thereof.

17. The polymer according to claim 15, wherein the at least one silicone monomer is present in an amount ranging from 20 to 90 wt. % relative to the total weight of the polymer.

18. The polymer according to claim 15, additionally comprising at least one carbon-containing monomer present in an amount ranging from 10 to 80 wt. % relative to the total weight of the composition.

19. The polymer according to claim 18, wherein the at least one carbon-containing monomer is chosen from:
methacrylates of formula CH₂═C(CH₃)—COOR1 wherein R1 is chosen from: linear or branched C₈ to C₂₂ alkyl groups; cyclic alkyl groups comprising from 8 to 30 carbon atoms; and tert-butyl groups;
acrylates of formula CH₂═CH—COOR2 wherein R2 is chosen from: linear or branched C₈ to C₂₂ alkyl groups; cyclic alkyl groups comprising from 8 to 30 carbon atoms; and isobutyl groups;
(meth)acrylamides of formulas CH₂═C(CH₃)—CONR3R4 and CH₂═CH—CONR3R4, wherein R3 is chosen from hydrogen atoms and linear or branched C₁ to C₁₂ alkyl groups, and R4 is chosen from linear or branched C₈ to C₁₂ alkyl groups;
di-n-alkyl itaconates of formula CH₂═C(CH₂—COO(CH₂)ₙ—CH₃)COO(CH₂)ₙ—CH₃, wherein n is an integer greater than or equal to 5;
vinyl esters of formula CH₂═CHOCOR5, wherein R5 is chosen from linear or branched from C₈ to C₂₂ alkyl groups;
vinyl ethers of formula CH₂═CH—OR6, wherein R6 is chosen from linear and branched alkyl groups, comprising from 8 to 22 carbon atoms;
linear or branched C₆ to C₂₂ alkyl (meth)acrylate homopolymers and copolymers, comprising at least one polymerizable end group chosen from vinyl and (meth)acrylate groups;
polyolefins comprising at least one end group which comprises at least one ethylenic unsaturation; and
mixtures thereof.

20. The polymer according to claim 19, wherein the at least one carbon-containing monomer is chosen from: the lauryl, behenyl, stearyl, isobornyl, and tert-butyl methacrylates; the lauryl, behenyl, 2-ethylhexyl, stearyl, isobornyl, isobutyl acrylates; macromonomers based on poly(ethyl-2-hexyl acrylate) and poly(dodecyl acrylate) comprising at least one mono(meth)acrylate end group; ethylene/butylene and ethylene/propylene macromonomers with at least one (meth)acrylate reactive end group; and mixtures thereof.

21. The polymer according to claim 15, additionally comprising at least one additional monomer chosen from:
(i) ethylenic hydrocarbons comprising from 2 to 10 carbons;
(ii) (meth)acrylates of formula CH₂═CHCOOR′₃ and CH₂═C(CH₃)COOR′₃ wherein R′₃ is chosen from:
linear or branched alkyl groups comprising from 1 to 7 carbon atoms, optionally interrupted by at least one heteroatom chosen from O, N, S and P; and optionally substituted with at least one substituent chosen from hydroxyl groups and halogen atoms;
C₃ to C₇ cycloalkyl groups,
C₃ to C₂₀ aryl groups,
C₄ to C₃₀ aralkyl groups (C₁ to C₈ alkyl group),
heterocyclic groups comprising from 4 to 12 ring members comprising at least one heteroatom chosen from O, N, and S, wherein the ring is aromatic or not aromatic,
heterocycloalkyl (C₁ to C₄ alkyl) groups,
and wherein said cycloalkyl, aryl, aralkyl, heterocyclic and heterocycloalkyl groups are optionally substituted with at least one substituent chosen from hydroxyl groups, halogen atoms, and linear or branched C₁₋₄ alkyl groups, said alkyl groups being optionally interrupted by at least one heteroatom chosen from O, N, S and P, and optionally substituted with at least one substituent chosen from hydroxyl groups and halogen atoms,
—(C₂H₄O)ₘ—R″ groups, wherein m is an integer ranging from 5 to 150 and R″ is chosen from H and from C₁ to C₃₀ alkyl groups; and
isobutyl methacrylate and tert-butyl acrylate;
(iii) (meth)acrylamides of formula: 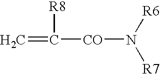
wherein R₈ is chosen from H and methyl; and R₇ and R₆, which may be identical or different, are chosen from:
hydrogen atoms,
linear or branched alkyl groups comprising 1 to 22 carbon atoms, said alkyl groups being optionally interrupted by at least one heteroatom chosen from O, N, S and P; and optionally substituted with at least one substituent chosen from hydroxyl groups and halogen atoms;
C₃ to C₁₂ cycloalkyl groups,
C₃ to C₂₀ aryl groups,
C₄ to C₃₀ aralkyl groups (C₁ to C₈ alkyl group),
heterocyclic groups comprising from 4 to 12 ring members comprising at least one heteroatom chosen from O, N, and S, wherein the ring is aromatic or not aromatic,
heterocycloalkyl (C₁ to C₄ alkyl) groups,
and wherein said cycloalkyl, aryl, aralkyl, heterocyclic and heterocycloalkyl groups are optionally substituted. with at least one substituent chosen from hydroxyl groups, halogen atoms, and linear or branched C₁-C₄ alkyl groups which are optionally interrupted by at least one heteroatom chosen from O, N, S and P, and optionally substituted with at least one substituent chosen from hydroxyl groups and halogen atoms;
(iv) vinylic compounds of formulas:
CH₂═CH—R₉, CH₂═CH—CH₂—R₉ and CH₂═C(CH₃)—CH₂—R₉
wherein R₉ is chosen from: hydroxyl groups; halogens; NH₂; OR₁₄ wherein R₁₄ is chosen from phenyl groups and from C₁ to C₇ alkyl groups; acetamide (NHCOCH₃); OCOR₁₅ groups wherein R₁₅ is chosen from linear or branched alkyl groups comprising from 2 to 7 carbon atoms; and/or a group chosen from:
linear or branched alkyl groups, comprising from 1 to 22 carbon atoms, wherein said alkyl groups are optionally interrupted by at least one heteroatom chosen from O, N, S and P; and optionally substituted with at least one substituent chosen from hydroxyl groups and halogen atoms,
C₃ to C₁₂ cycloalkyl groups,
C₃ to C₂₀ aryl groups,
C₄ to C₃₀ aralkyl groups (C₁-C₈ alkyl group),
heterocyclic groups comprising from 4 to 12 ring members comprising at least one heteroatom chosen from O, N, and S, wherein the ring is aromatic or not aromatic,
heterocycloalkyl (C₁ to C₄ alkyl) groups,
and wherein said cycloalkyl, aryl, aralkyl, heterocyclic and heterocycloalkyl groups optionally comprise at least one substituent chosen from hydroxyl groups, halogen atoms, and linear and branched alkyl groups comprising from 1 to 4 carbon atoms, said alkyl groups being optionally interrupted by at least one heteroatom chosen from O, N, S and P, and optionally substituted with at least one substituent chosen from hydroxyl groups and halogen atoms;
(v) monomers comprising at least one ethylenic unsaturation which comprises at least one carboxylic, phosphoric and/or sulphonic acid function, and/or anhydride, and salts thereof,
(vi) monomers comprising at least one ethylenic unsaturation which comprises at least one tertiary amine function and salts thereof; and
(vii) mixtures thereof.

22. The polymer according to claim 21, wherein the at least one additional monomer is chosen from (meth)acrylic acid, from C₁ to C₄ alkyl and from C₃ to C₇ cycloalkyl (meth)acrylates, isobutyl methacrylate, tert-butyl acrylates, acrylic acid and methacrylic acid.

23. The polymer according to claim 15, wherein said at least one first and at least one second blocks are linked together by at least one intermediate block or sequence comprising at least one monomer constituting said first block and at least one monomer constituting said second block.

24. A method of preparation of a polymer by radical solution polymerization comprising:
heating at least one polymerization solvent in a reactor to a temperature suitable for polymerization;
then after reaching said temperature, adding at least one monomer constituting the first block in the presence of a quantity of at least one polymerization initiator,
then waiting a time T for conversion of the monomers constituting the second block,
then adding another quantity of said at least one polymerization initiator,
then allowing the mixture to react for a time T′,
then returning the mixture to room temperature, wherein said polymer is a block (co)polymer comprising at least one first block and at least one second block, wherein at least one block comprises at least one silicone monomer in an amount ranging from 35 to 100 wt. % relative to the total weight of said block, wherein said at least one silicone monomer is chosen from:
(i) ethylenic monomers comprising at least one ester group which comprises at least one silane and/or siloxane, of formula: 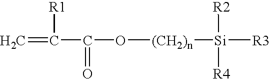
wherein:
R1 is chosen from H and methyl;
R2, R3, R4, which may be identical or different, comprise C₁ to C₆ alkyl groups and/or —OSi(R5)₃ groups, wherein R5 is chosen from methyl and ethyl; and
n is an integer ranging from 1 to 10;
(ii) tPDMS macromonomers and polydimethylsiloxanes comprising at least one monoacryloyloxy and/or monomethacryloyloxy end group of the following formula 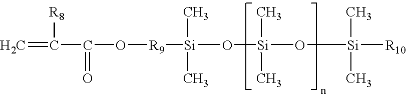
wherein:
R8 is chosen from hydrogen atoms and methyl groups;
R9 is chosen from linear or branched divalent hydrocarbon groups comprising from 1 to 10 carbon atoms and optionally comprising one or two ether bonds —O—;
R10 is chosen from a linear or branched alkyl groups, comprising from 1 to 10 carbon atoms; and
n is an integer ranging from 1 to 300.
(iii) ethylenic monomers comprising at least one ester group which comprises at least one dendrimeric carboxysilane of formula: 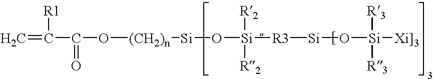
wherein:
R1 is chosen from Hand methyl;
n is an integer ranging from 1 to 10;
R′2, R″2, R′3 and R″3, which may be identical or different, comprise at least one C₁ to C₁₀ alkyl group;
R3 comprises at least one from C₂ to C₁₀ divalent alkylene group;
i is an integer ranging from 1 to 10; and
when i is an integer ranging from 2 to 10, X(i), which may be identical or different, comprises at least one —R4-Si—[O—(R′3)(R″3)-X(i−1)]₃ group, wherein R4, which may be identical or different, comprises at least one from C₂ to C₁₀ divalent alkylene group; and
when i is 1, X(1) is chosen from H and from C₁ to C₁₀ alkyl groups;
(iv) ethylenic monomers of type POSS and POS of structure: 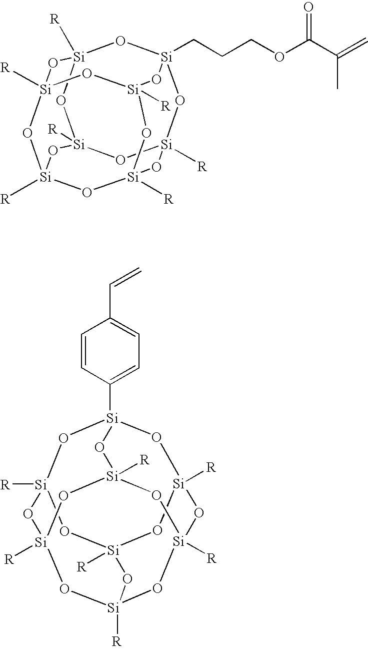
wherein R, which may be identical or different, is chosen from C₁ to C₁₀ linear and from C₃ to C₁₂ cyclic alkyl groups; and
(v) mixtures thereof.
